(19) United States
(12) Patent Application Publication
(10) Pub. No.: US 2008/0118932 A1
(43) Pub. Date: May 22, 2008

(54) METHODS OF ANALYZING GLYCOMOLECULES

(75) Inventors: Kelly N. Toler, Chelmsford, MA (US); Tanya Q. Shang, Malden, MA (US); Thomas J. Porter, Reading, MA (US); Jason C. Rouse, Londonderry, NH (US)

Correspondence Address: FISH & RICHARDSON PC, P.O. BOX 1022, MINNEAPOLIS, MN, 55440-1022, US

(73) Assignee: WYETH

(21) Appl. No.: 11/937,778

(22) Filed: Nov. 9, 2007

Related U.S. Application Data
(60) Provisional application No. 60/857,833, filed on Nov. 9, 2006.

(51) Int. Cl. G01N 33/53; G01N 33/92

(52) U.S. Cl. 435/7.1; 436/71

(57) ABSTRACT

The invention relates to methods for analyzing glycomolecules such as glycoproteins, and glycan structures associated with preparations of such glycomolecules.

### CROSS-REFERENCE TO RELATED APPLICATIONS

This application claims priority to U.S. Application Ser. No. 60/857,833, filed on Nov. 9, 2006. The disclosures of the prior application is considered part of (and is incorporated by reference in) the disclosure of this application.

### TECHNICAL FIELD

The invention relates to methods for analyzing glycomolecules such as glycoproteins, and glycan structures associated with preparations of such glycomolecules.

### BACKGROUND

Characterization and routine analysis of glycosylation patterns in glycoprotein therapeutics is important for the assessment of product quality and consistency of manufacture in the development and commercialization of protein pharmaceuticals. R. J. Harris et al., Drug Dev. Res. 61 (2004) 137-154, J. M. Coco-Martin, BioProcess Int. 2 (2004) 32-40, R. Jefferis, Biotechnol. Prog. 21 (2005) 11-16 and N. Jenkins et al., Nat. Biotechnol. 14 (1996) 975-981. Commonly, the N-linked oligosaccharides are characterized and monitored through enzymatic release from the mAb with an endoglycosidase and subsequent separation of the individual native or fluorescently labeled structures by chromatographic (M. Weitzhandler, et al., J. Pharm. Sci. 83 (1994) 1670-1675; F. H. Routier et al., Glycoconjugate J. 14 (1997) 201-207; A. E. Hills et al., Biotechnol. Bioeng. 75 (2001) 239-251; J. Charlwood et al., Proteomics 1 (2001) 275-284; J. Siemiatkoski et al., Carbohydrate Res. 341 (2006) 410-419) or electrophoretic methods (T. S. Raju et al., Glycobiology, 10 (2000) 477-486; S. Ma et al., Anal. Chem. 71 (1999) 5185-5192; Y. Mechref et al., Electrophoresis 10 (2005) 2034-2046).

Traditionally, released N-glycan pools from recombinant proteins (L. J. Basa et al., J. Chromatogr. A 499 (1990) 205-220) and monoclonal antibodies (M. Weitzhandler et al., J. Pharm. Sci. 83 (1994) 1670-1675) were profiled directly by high-pH anion-exchange chromatography (HPAEC) with pulsed amperometric detection (PAD). More recently, detection methods for carbohydrates have been developed that utilize separation by typical HPLC methods, with or without carbohydrate labeling. G. R. Guile et al., Anal. Biochem. 240 (1996) 210-226; K. R. Anumula et al., Glycobiology 8 (1998) 685-694; N. Takahashi et al., Anal Biochem. 226 (1995) 139-146.

Detailed structural characterization of N-glycans at the low to mid picomole level can be obtained through the combined use of chromatography, MS, exo- and endoglycosidases, and bioinformatics. P. M. Rudd et al., Curr. Opin. Biotech. 8 (1997) 488-497.

### SUMMARY

The analysis of glycomolecule preparations such as glycoprotein preparations, e.g., preparations of antibodies or antigen-binding fragments thereof, by a combination of analytical approaches can be used to evaluate glycan structures associated with the glycomolecule preparation. Such methods can be used, e.g., to ensure that the glycomolecule has a preselected property. For example, using the methods described herein, a glycomolecule can be analyzed or processed to determine whether it meets a predetermined condition or reference standard, e.g., a release specification, a compendial requirement, a regulatory requirement, e.g., a label requirement. The methods can also be used to evaluate, e.g., what effect differences in production procedures for a glycomolecule may have on glycan structure. Other approaches for applying the disclosed methods are provided herein.

In particular, methods are described that provide for analysis or processing of sialylated, e.g., di-sialylated and mono-sialylated, and non-sialylated, complex glycans, high mannose glycans and hybrid glycans. Such methods provide compatible conditions for analysis or processing of N-glycan structures by liquid chromatography (LC), e.g., high performance liquid chromatography (HPLC) and mass spectrometry (MS), e.g., without intermediate processing steps such as drying, ion pairing reagent removal, desalting or dialysis, between LC and MS analysis.

Accordingly, in one aspect, the invention features a method of evaluating or processing a glycan structure or structures of a glycomolecule preparation. The method includes: subjecting a glycan or glycans from a glycomolecule preparation to LC, e.g., HPLC, in the absence of a salt or an ion paring agent to evaluate a glycan structure or structures, and subjecting the HPLC-evaluated, separated or purified glycan or glycans to mass spectrometry to further evaluate a glycan structure or structures, to thereby evaluate or process the glycan structure or structures of a glycomolecule preparation.

In one embodiment, the glycan structure or structures is an N-linked glycan, an O-linked glycan, or combinations thereof. The N-linked glycan structure or structures can be, e.g., one or more of: a sialylated, complex glycan, e.g., a di-sialylated glycan, a mono-sialylated glycan, or combinations thereof, a non-sialylated complex glycan, a high mannose glycan, a hybrid glycan or combinations thereof. In one embodiment, the complex sialylated or non-sialylated complex glycan can be monoantennary, biantennnary, triantennary or tetraantennary. Preferably, the complex sialylated or non-sialylated complex glycan is monoantenarry and biantennnary.

In one embodiment, the glycomolecule preparation is selected from the group consisting of a glycoprotein preparation, a glycopolypeptide preparation and a glycolipid preparation. In one embodiment, the glycomolecule preparation is a preparation of an antibody or antigen binding fragment thereof.

In one embodiment, prior to LC, the glycan or glycans are removed, e.g., chemically or enzymatically removed, from the glycomolecule. In one embodiment, the glycan is enzymatically removed using an endoglycosidase, an exoglycosidase or a combination thereof. Examples of endoglycosidases for N-glycan removal include PNGase F and endoH. An example of an enzyme for O-glycans is endo-N-acetylgalactosaminidase (O-glycanase). In one embodiment, the glycan or glycans can be labeled, e.g., prior to analysis with LC. For example, the glycan or glycans from a preparation can be labeled with a fluorescent label or a radioisotope. Examples of fluorescent labels include, e.g., 2-aminobenzamide (2-AB), 2-aminopyridine (PA), and anthranilic acid (such as 2-anthranilic acid, 2-AA).

In one embodiment, the LC-evaluated, separated or purified glycan or glycans are not subjected to one or more of ion pairing removal, desalting, dialysis and drying prior to evaluation with mass spectrometry or with sufficiently little ion pairing agent or agents such that the LC fractions can be subjected to mass spectrometry, e.g., electrospray ionization mass spectrometry (ESI-MS) or matrix assisted laser desorption/ionization mass spectrometry (MALDI-MS) without a step of removal of the ion pairing agent or agents.

In one embodiment, the LC is HPLC and the HPLC is selected from hydrophilic interaction chromatography, e.g., normal phase HPLC (NP-HPLC), and reverse phase HPLC (RP-HPLC). In one embodiment, the HPLC is NP-HPLC and the HPLC uses a polySulfoethyl Aspartamide (polySEA) column or a SeQuant column. In one embodiment, the columns includes one or more of the following functional groups: carbamoyl groups, sulfopropyl groups, sulfoethyl groups (e.g., poly (2-sulfoethyl aspartamide)), hydroxyethyl groups (e.g., poly (2-hydroxyethyl aspartamide)) and aromatic sulfonic acid groups. Preferred functional groups include sulfoethyl groups such as poly (2-sulfoethyl aspartamide) and sulfopropyl groups such as CH₂N(CH₃)₂CH₂CH₂CH₂SO₃.

In one embodiment, the glycan or glycans are subjected to HPLC with a mobile phase of acetonitrile, water or a combination thereof. Preferably, the glycan or glycans are subjected to HPLC with a mobile phase of acetonitrile and water.

In one embodiment, the mass spectrometry is one or more of: electrospray ionization mass spectrometry (ESI-MS), turbospray ionization mass spectrometry, nanoelectrospray ionization mass spectrometry, microelectrospray ionization mass spectrometry, sonic spray ionization mass spectrometry and matrix assisted laser desorption/ionization mass spectrometry (MALDI-MS). Preferably, the mass spectrometry is ESI-MS, MALDI-MS or a combination of ESI-MS and MALDI-MS. In one embodiment, the evaluation by mass spectrometry uses a quadrupole mass analyzer, a time of flight (TOF) mass analyzer, a hybrid quadrupole/linear ion trap mass analyzer, a hybrid linear ion trap/orbitrap mass analyzer, a hybrid linear ion trap/FT (Fourier transform) mass analyzer, hybrid quadrupole/FT mass analyzer or a hybrid quadrupole/time of flight (Q-TOF) mass analyzer.

In one embodiment, the glycan structure or structures are analyzed by LC and/or MS for branching, linkages between monosaccharides and location of monosaccharides. In one embodiment, the glycomolecule preparation is evaluated for the presence or quantity of: a fucosylated biantennary complex glycan having no non-reducing end terminal galactose residues, a fucosylated biantennary complex glycan having one non-reducing end terminal galactose residue, a fucosylated biantennary complex glycan having two non-reducing end terminal galactose residues, a biantennary complex glycan having no non-reducing end terminal galactose residues, a biantennary complex glycan having one non-reducing end terminal galactose residue, a biantennary complex glycan having two non-reducing end terminal galactose residues, a fucosylated biantennary complex glycan having two galactose residues and one N-acetylneuraminic acid residue, a fucosylated biantennary complex glycan having two galactose residues and two N-acetylneuraminic acid residues, a biantennary complex glycan having two galactose residues and two N-acetylneuraminic acid residues, a high mannose glycan having five mannose residues, a high mannose glycan having six mannose residues, a high mannose glycan having seven mannose residues, a high mannose glycan having eight mannose residues, and a high mannose glycan having nine mannose residues.

In one embodiment, the method includes evaluating by LC for a value for a glycan structure or structures to determine if the value meets a reference standard. The value can be the presence or amount of a glycan structure or structures.

In one embodiment, the method includes evaluating by MS for a value for a glycan structure or structure to determine if the value meets a reference standard. The value can be the presence of a glycan structure or structures.

In one embodiment, the method includes evaluating by LC for a value for a glycan structure or structures to determine if the value meets a reference standard and evaluating by MS for a value for a glycan structure or structures to determine if the value meets a reference standard. The value evaluated by LC can be the presence or amount of a glycan structure or structures. The value evaluated by MS can be the presence of a glycan structure or structures.

In one embodiment, the method includes determining if the test value for a glycan structure or structures obtained by LC is equal to or greater than a reference standard, if it is less than or equal to a reference standard, or if it falls within a range. In other embodiments, a test value, e.g., obtained by LC or MS, need not be a numerical value but may merely indicate whether the glycan structure or structures are present.

In preferred embodiments, the test value can be memorialized, e.g., in a computer readable record. The reference standard can be, e.g., a release specification, a compendia specification, a regulatory required specification, e.g., a label specification. In some embodiments, the reference standard is a different preparation of the glycomolecule, e.g., a reference standard prepared by a different process than the glycomolecule being evaluated. For example, the reference standard can be prepared by a different manufacturing process, e.g., culture and/or isolation step, or different expression system.

In one embodiment, the method further includes making a decision about the glycomolecule preparation based upon the analysis. The decision can be, e.g., one or more of accepting or discarding the preparation, releasing or withholding the preparation, formulating the preparation, packaging the preparation, labeling the preparation, shipping, relocating, selling or offering to sell the preparation.

In one embodiment, the method further includes evaluating a second parameter of the glycomolecule preparation, e.g., a physical parameter, e.g., molecular weight of the glycomolecule or fragments thereof, isoelectric point, protein or lipid composition, a biological property (e.g., binding affinity, antigen specificity). The method can include comparing the evaluation of the second parameter with a reference standard for that parameter.

In one embodiment, the glycomolecule is a glycoprotein and the glycoprotein preparation has further been evaluated by one or more of peptide mapping and peptide sequencing. In one embodiment, the glycoprotein has been digested and the digested peptide fragments have been evaluated using HPLC, e.g., RP-HPLC, and mass spectrometry, e.g., ESI-MS. In one embodiment, the glycoprotein has been digested and the digested peptide fragments have been evaluated using MS/MS, e.g., nanoESI-q-TOF MS/MS. The glycoprotein can be digested, e.g., by reduction/alkylation and proteolysis.

In one embodiment, the glycomolecule is a glycoprotein and the glycoprotein preparation has further been evaluated by one or more of intact and subunit analysis. In one embodiment, the glycan has been removed from the glycoprotein and the protein has been evaluated using HPLC, e.g., RP-HPLC, and mass spectrometry, e.g., ESI-q-TOF MS. In one embodiment, the glycan has been removed from the glycoprotein and the protein has been evaluated using ESI-MS, e.g., nanoESI-q-TOF. The glycan can be removed, e.g., by methods described herein. In one embodiment, the glycoprotein is an antibody and heavy chain and light chain subunits of the glycoprotein preparation are separated, e.g., using reduction and alkylation. In one embodiment, the heavy chain and/or light chain subunits of the glycoprotein preparation have been evaluated using HPLC, e.g., RP-HPLC, and mass spectrometry, e.g., ESI-Q-TOF MS. In one embodiment, the heavy chain and/or light chain subunits of the glycoprotein preparation have been evaluated using ESI-MS, e.g., nanoESI-Q-TOF.

In one embodiment, the glycomolecule preparation is a test batch and the test batch can be evaluated to determine if the test preparation is expected to have one or more properties of a glycomolecule preparation, e.g., a commercially available version of the glycomolecule preparation.

In one aspect, the invention features a method of evaluating or processing a glycomolecule preparation that includes providing a determination about a glycomolecule preparation based upon based upon a method described herein. The method can further include accepting or discarding the preparation, releasing or withholding the preparation, formulating the preparation, packaging the preparation, labeling the preparation, shipping, relocating, selling or offering to sell the preparation, based upon the determination. In one embodiment, the party making the determination is not the party that provided the analysis by a method described herein.

In one aspect, the invention features a method of determining if a glycomolecule preparation meets a reference standard, e.g., a release standard, a compendial specification or a regulatory standard, e.g., a label requirement. The method includes providing a value for a glycan structure or structures by a method described herein, and comparing the test value to a reference standard, to determine if the glycomolecule preparation meets the standard.

In another aspect, the invention features a method of evaluating a glycomolecule preparation for a biological activity that includes proving an evaluation of a glycan structure or structures of the glycomolecule preparation obtained by a method described herein, and comparing the evaluation of the glycan structure or structures to an evaluation obtained by a method described herein on a second glycomolecule preparation, and making a determination regarding biological activity of the glycoprotein preparation based upon similarities or differences in the glycan structure or structures of the glycomolecule preparation and the second glycomolecule preparation.

In one embodiment, the glycan structure or structures are a direct measure of the biological activity. In another embodiment, the glycan structure or structures are an indirect measure of biological activity.

Examples of biological activities that can be evaluated include immunogenecity, half life, stability, clearance, binding specificity and binding affinity.

In one aspect, the invention features a method of evaluating the effect of glycan structures or structures on a biological activity of a glycomolecule. The method includes providing a first glycomolecule preparation having a first activity or level of an activity and a second glycomolecule preparation that does not have the activity or has a different level of the activity, providing an evaluation of glycan structure of the first glycomolecule preparation and the second glycomolecule preparation obtained by the method described herein and determining the absence or presence of differences in the glycan structure or structures of the first and second preparation, to thereby evaluate the effect of the glycan structure or structures on the activity.

In one aspect, the invention features a method of evaluating or processing a glycomolecule preparation that includes: providing a value for a glycan structure or structures of a glycomolecule preparation by a method described herein, wherein the glycomolecule preparation was made under a first set of conditions, providing a value for a glycan structure or structures of a glycomolecule preparation by a method described herein, wherein the glycomolecule preparation was made under a second set of conditions, and comparing the value for the glycomolecule preparation made under the first conditions to the value of the glycomolecule preparation made under the second set of conditions, or comparing the values for the glycomolecule preparations made under the first and/or second set of conditions to a reference standard. Such methods allow analysis of the effect, if any, of changes in process, e.g., manufacturing process, culture, isolation/[purification, expression systems, on glycan structure.

In one aspect, the invention features a method of analyzing a process, e.g., a manufacturing process, of a glycomolecule preparation that includes providing a glycomolecule preparation made by a selected process, analyzing a value for a glycan structure or structures of the glycomolecule preparation by a method described herein, and comparing the value to a reference standard, to thereby evaluate the process.

In one embodiment, the glycomolecule preparation is prepared by the same process as the process used to obtain the glycomolecule preparation or preparations used to obtain the reference standard. In another embodiment, the glycomolecule preparation is made by a different process than the glycomolecule preparation or preparations used to obtain the reference standard.

In one embodiment, the method further includes maintaining the manufacturing process based, at least in part, upon the analysis. In another embodiment, the method includes altering the manufacturing process based, at least in part, upon the analysis.

Methods described herein are useful for analyzing or processing a glycomolecule preparation, e.g., to determine whether to accept or reject a batch of the glycomolecule preparation, or to guide a step in the production of a glycomolecule preparation.

A new NP-HPLC method for monoclonal N-glycan analysis that is fully compatible with on-line MS has been developed. When coupled on-line with a QTOF mass spectrometer, this method facilitates sensitive characterization of carbohydrate structures via accurate mass measurements. Full MS compatibility of the NP-HPLC method is demonstrated by the close agreement of the TIC and the fluorescence profile for each mAb N-glycan sample examined. Additionally, the N-glycan identities and relative abundances detected by this method are consistent with tryptic glycopeptide mapping and the mass analysis of intact light and heavy chain subunits for a specific mAb sample, which demonstrates the on-line NP-HPLC/MS method provides comprehensive analysis of the mAb carbohydrates.

Given that ion pairing reagents or salts are not used in the NP-HPLC method, minor peaks in the N-glycan profile can be collected, concentrated, and analyzed off-line by MALDI MS without further purification. This enables straightforward structural characterization by MALDI-QTOF MS/MS through dissociation of the [M+Na]⁺ carbohydrate ions. The reliable fragment ion abundance data in the MS/MS spectra obtained with MALDI-QTOF instrumentation permits a knowledge-based approach that can be used to confirm the identification of oligosaccharides based on the accurate mass data obtained from LC/MS. The identities of several mAb N-glycans were confirmed by comparing the MS/MS spectra of standards and those of carbohydrates from the NP-HPLC profiles. Furthermore, MALDI-QTOF MS/MS can be applied to distinguish biantennary N-glycan isomers differing in the location of a single residue at the non-reducing termini (e.g. G1F isomers) or determine whether a particular monosaccharide is located at the reducing or non-reducing terminus (e.g. G0-GlcNAc isomers). These results show that the knowledge-based characterization strategy with the appropriate training set (i.e. collection of MS/MS spectra of closely related structures) is useful in the identification of unknown species in the N-glycan profiles. Additionally, the knowledge-based strategy and the on-line NP-HPLC/MS method can be applied in the analysis of glycans released by endoglycosidases other than PNGase F or combined with exoglycosidase sequencing to assist in the elucidation of carbohydrate structures.

The on-line NP-HPLC/MS method described herein is effective in characterizing mAb N-glycans with complex-type asialobiantennary structures and can be easily integrated into top-down and bottom-up mAb characterization strategies. The reproducibility of the chromatographic profiles and mass spectra from the NP-HPLC/MS method allows application of this method in comparability studies of mAb N-glycan distributions. Although mAb N-glycans primarily are asialo-complex species, the NP-HPLC method for the separation of oligomannose structures released from a recombinant glycoprotein is quite useful. However, the general methodology of developing a chromatographic method using mobile phases that are compatible with MS can be applied to discover an on-line LC/MS method for analysis of heavily sialylated, complex-type N-glycans.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a depiction of NP-HPLC chromatograms of commercially obtained 2AB derivatized N-glycan standards. Each standard (˜100 pmol) was injected separately. The oligomannose N-glycans are designated as Manx, where x indicates the number of mannose residues.

FIG. 2 is a display of data from NP-HPLC/MS of 2AB derivatized N-glycans from an IgG1 mAb. Reference standard are shown including the fluorescence profile (A) and TIC (B) as well as corresponding mass spectra and relative mass errors for some minor (C through E) and major (F) N-glycans. The relative percentages of the major N-glycans from the fluorescence profile are indicated in A and confirmed fluorescence peak assignments are labeled in B. The asterisks in the TIC represent underivatized, non-reduced (green) and reduced (orange) G0F, G1F, and G2F, respectively.

FIG. 3 is a depiction of a comparison of the MALDI-QTOF MS/MS spectra of sodiated G2F from a commercially available standard (A) and a fraction collected from the N-glycan profile of an IgG4 mAb reference standard (B). These MS/MS spectra were obtained at a CE of 110 eV.

FIG. 4 is a display of MALDI-QTOF MS/MS spectra of (A) G1Fa and (B) G1Fb isomers that were initially collected from the N-glycan profile of an IgG4 mAb reference standard, separately rechromatographed, and collected prior to MS/MS. The MS/MS experiments were performed with a CE of 100 eV.

FIG. 5 is a demonstration of the reproducibility of the fragment ion abundances in the MALDI-QTOF MS/MS spectra of G1Fa and G1Fb isomers collected from the N-glycan profiles of various mAbs: (A,B) an IgG4 reference standard, (C,D) an IgG1 reference standard, and (E,F) an IgG4.

FIG. 6 is a depiction of NP-HPLC profiles of N-glycans released from an IgG1 mAb using PNGase F from different sources: (A) recombinant source and (B) native source. The inserts show minor variation in the profiles prior to the elution of G0F. Mass spectra corresponding to minor Man5 related peaks tentatively labeled as Man5 in (A) and Man5-GlcNAc in (B).

FIG. 7 is a depiction of a knowledge-based characterization of the Man5 related species of FIG. 6 via comparison of the MALDI-QTOF MS/MS spectra of: (A) a commercially available Man5 standard (CE of 85 eV), (B) the Man5 mAb sample (CE of 85 eV), and (C) the Man5-GlcNAc mAb sample (CE 70 eV). The insert in (C) demonstrates the resolution of two fragment ions, one of which (Y_{2α}) is unique to the Man-5-GlcNAc carbohydrate.

FIG. 8 is a depiction of knowledge-based characterization of the G0-GlcNAc isomers of FIG. 6 via comparison of the MALDI-QTOF MS/MS spectra of: (A) a commercially available G0 standard (CE of 83 eV), (B) the G0-GlcNAc (22.2 min.) fraction from the profile in FIG. 6C (CE of 74 eV), and (C) the G0-GlcNAc (23.3 min.) fraction from the profile in FIG. 6C (CE of 74 eV). Knowledge-based characterization of the G0F-GlcNAc species of FIG. 6 via comparison of the MALDI-QTOF MS/MS spectra of: (D) a commercially available G0F standard (CE of 93 eV) and (E) the G0F-GlcNAc fraction from the profile in FIG. 6B (CE of 80 eV).

FIG. 9 depicts N-glycans commonly observed for monoclonal antibodies derived from Chinese hamster ovary (CHO) cell expression systems.

### DETAILED DESCRIPTION

Methods described herein provide analytical techniques for evaluating glycans associated with glycomolecule preparations such as glycoprotein preparations, glycopolypeptide preparations, and glycolipid preparations. For example, methods disclosed herein can be used to evaluate or process a preparation of an antibody or antigen-binding fragment thereof.

A “glycomolecule” as used herein refers to a molecule that includes a glycan moiety and a non-glycan moiety. The non-glycan moiety can be, e.g., a protein, polypeptide or lipid.

The term “protein” as used herein refers to one or more polypeptides that can function as a unit. The term “polypeptide” as used herein refers a sequential chain of amino acids linked together via peptide bonds. The term “polypeptide” is used to refer to an amino acid chain of any length, but one of ordinary skill in the art will understand that the term is not limited to lengthy chains and can refer to a minimal chain comprising two amino acids linked together via a peptide bond. If a single polypeptide can function as a unit, the terms “polypeptide” and “protein” may be used interchangeably.

The term “antibody” refers to any immunoglobulin or fragment thereof, and encompasses any peptide or polypeptide comprising an antigen-binding site. The term includes, but is not limited to, polyclonal, monoclonal, monospecific, polyspecific, bispecific, humanized, de-immunized, human, camelid, rodent, single-chain, chimeric, synthetic, recombinant, hybrid, mutated, grafted, and in vitro generated antibodies. The term “antibody” also includes antibody fragments and variant molecules such as Fab, F(ab′)₂, Fv, scFv, Fd, dAb, VHH, and other antibody fragments and variant molecules that retain antigen-binding function. Typically, such fragments would comprise an antigen-binding domain.

The term “preparation” refers to any composition containing at least one glycosylated molecule, for example, at least one glycoprotein, at least one glycopolypeptide or at least one glycolipid.

Various proteins and polypeptides that can be analyzed by the disclosed methods are described in detail below.

### Analysis of Glycan Structure

Methods disclosed herein provide for analysis or processing of glycans, e.g., N-linked or O-linked glycans. Proteins and polypeptides can be glycosylated at arginine residues, referred to as N-linked glycosylation or N-linked glycans, and at serine or threonine residues, referred to as O-linked glycosylation or O-linked glycans. Exemplary N-linked linked glycans include complex, high mannose and hybrid glycans. Complex, high mannose and hybrid glycans can result from different processing events to a precursor oligosaccharide that can occur in a cell. A “precursor oligosaccharide” as used herein refers to the oligosaccharide chain involved in the initial steps in biosynthesis of carbohydrate chains. A “precursor oligosaccharide” can be an oligosaccharide structure which includes at least the following sugars: Man₉GlcNAc₂, for example, a precursor oligosaccharide can have the following structure: Glc₃Man₉GlcNAc₂. High mannose glycans can include three to nine mannose residues attached to GlcNAc₂. Exemplary high mannose glycans that can be evaluated by the methods described herein include: a high mannose glycan having five mannose residues (also referred to as “Man5”), a high mannose glycan having six mannose residues (also referred to as “Man6”), a high mannose glycan having seven mannose residues (also referred to as “Man7”), a high mannose glycan having eight mannose residues (also referred to as “Man 8”), and a high mannose glycan having nine mannose residues (also referred to as “Man9”).

Complex glycans include a pentasaccharide core of Man₃GlcNAc₂, which can be fucosylated or non-fucosylated, and may contain two, three or four outer branches (“antennae”) attached to pentasaccharide core. These structures are referred to in terms of the number of their outer branches: biantennary (two branches), triantennary (three branches) or tetraantennary (four branches). Exemplary complex glycans that can evaluated by the methods disclosed herein include: a fucosylated biantennary complex glycan having no reducing end terminal galactose residues (also referred to herein as “G0F”), a fucosylated biantennary complex glycan having one reducing end terminal galactose residue (also referred to herein as “G1F”), a fucosylated biantennary complex glycan having two reducing end terminal galactose residues (also referred to as “G2F”), a non-fucosylated biantennary complex glycan having no reducing end terminal galactose residues (also referred to herein as “G0”), a non-fucosylated biantennary complex glycan having one reducing end terminal galactose residue (also referred to herein as “G1”), a non-fucosylated biantennary complex glycan having two reducing end terminal galactose residues (also referred to herein as “G2”), a fucosylated biantennary complex glycan having two galactose residues and one N-acetylneuraminic acid residue (also referred to herein as “G2F/NeuAc”), a fucosylated biantennary complex glycan having two galactose residues and two N-acetylneuraminic acid residues (also referred to as “G2F/2NeuAc”) and any of these structures having one or two terminal sialic acid residues. In addition, isomers of complex glycans having a monosaccharide residue present on one chain but not on another can be analyzed by the methods described herein.

Hybrid glycans can include one or more high mannose branch and one or more complex glycan branch. Again, isomers of a hybrid glycan can also be analyzed by methods disclosed herein.

Specifically, methods disclosed herein can provide compatible conditions for analysis or processing of glycan structures by high performance liquid chromatography (HPLC) and mass spectrometry (MS), e.g., without intermediate processing steps such as drying, ion pairing reagent removal, desalting or dialysis, between HPLC and MS analysis. The methods disclosed herein can include determining a value for a glycan structure or structure using, e.g., LC and/or MS, a comparing that value to a reference standard. A reference standard, by way of example, can be a value determined from a reference sample (e.g., a commercially available preparation or a previous production (e.g., batch or batches) of the preparation. The reference standard can be whether a glycan structure or structure is present or absent. In other embodiments, the reference standard can be the amount of one or more glycan structures in a preparation. Other examples of reference standards can be release standards (e.g., requirements which should be meet for commercialization) or production requirements required, e.g., by third parties such as a regulatory authority, e.g., compendial requirements or label requirements.

Removal of Glycans from the Glycomolecule

The glycan portion of the glycomolecule can be removed prior to analysis with HPLC and mass spectrometry. The glycan can be removed chemically of enzymatically. For example, N— and O— glycans can be chemically removed by one or more of: hydrazinolysis, β-elimination, and hydrogen fluoride treatment.

For enzymatic removal of glycans, various endoglycosidases and exoglycosidases can be used. For example, endoglycosidases such as peptide-N-glycosidase F (PNGase F) and endoglycosidase H (endoH) can be used to remove N-linked glycans from a glycomolecule. Endoglycosidases, both in recombinant and native forms, are commercially available. For example, native PNGase F is commercially available from New England BioLabs (Beverly, Mass.) and recombinant PNGase F is available from Prozyme (San Leandro, Calif.). Preferred conditions for glycan removal by enzymatic digestion include overnight digestion (e.g., of about 16 hours or more) at 37° C.

Derivation of Glycans

Prior to analysis of the glycan or glycans by HPLC and mass spectrometry, the glycans can be labeled. Preferably, the analyte, e.g., glycans, can be fluorescently labeled (derivatized), which can aid in sensitivity and/or selectivity. Examples of fluorescent labels include 2-aminobenzamide (2-AB), 2-aminopyridine (PA), and anthranilic acid (such as 2-anthranilic acid, 2-AA). The analyte can be labeled using techniques known in the art (see e.g., Bigge et al., Anal. Biochem. 230:229-238 (1995); Oxford GlycoSciences Signal Labeling Kit). A preferred label is 2-AB. In other embodiments, the analyte can be radiolabeled (derivatized), e.g., tritium labeled.

High Performance Liquid Chromatography (HPLC)

Liquid chromatography, including HPLC, can be used to analyze structures, such as glycans (e.g., N-glycans and/or O-glycans), that can be present, for example, on a protein. The glycan can released, e.g., as described herein, from the molecule prior to analysis. Various forms of LC can be used to study these structures, including anion-exchange chromatography, reversed-phase HPLC, size-exclusion chromatography, high-performance anion-exchange chromatography, and normal phase (NP) chromatography, including NP-HPLC (see, e.g., Alpert et al., J. Chromatogr. A 676:191-202 (1994)). Hydrophilic interaction chromatography (HILIC) is a variant of NP-HPLC that can be performed with partially aqueous mobile phases, permitting normal-phase separation of peptides, carbohydrates, nucleic acids, and many proteins. The elution order for HILIC is least polar to most polar, the opposite of that in reversed-phase HPLC. HPLC can be performed, e.g., on an HPLC system from Waters (e.g., Waters 2695 Alliance HPLC system), Agilent, Perkin Elmer, Gilson, etc.

NP-HPLC, preferably HILIC, is a particularly useful form of HPLC that can be used in the methods described herein. NP-HPLC separates analytes based on polar interactions between the analytes and the stationary phase (e.g., substrate). The polar analyte associates with and is retained by the polar stationary phase. Adsorption strengths increase with increase in analyte polarity, and the interaction between the polar analyte and the polar stationary phase (relative to the mobile phase) increases the elution time. Use of more polar solvents in the mobile phase will decrease the retention time of the analytes while more hydrophobic solvents tend to increase retention times.

Various types of substrates can be used with NP-HPLC, e.g., for column chromatography, including silica, amino, amide, cellulose, cyclodextrin and polystyrene substrates. Examples of useful substrates, e.g., that can be used in column chromatography, include: polySulfoethyl Aspartamide (e.g., from PolyLC), a sulfobetaine substrate, e.g., ZIC®-HILIC (e.g., from SeQuant), POROS® HS (e.g., from Applied Biosystems), POROS® S (e.g., from Applied Biosystems), PolyHydroethyl Aspartamide (e.g., from PolyLC), Zorbax 300 SCX (e.g., from Agilent), PolyGLYCOPLEX® (e.g., from PolyLC), Amide-80 (e.g., from Tosohaas), TSK GEL® Amide-80 (e.g., from Tosohaas), Polyhydroxyethyl A (e.g., from PolyLC), Glyco-Sep-N (e.g., from Oxford GlycoSciences), and Atlantis HILIC (e.g., from Waters). Preferred columns include polySulfoethyl Aspartamide and ZIC®-HILIC; the most preferred column being polySulfoethyl Aspartamide. Column that can be used in the disclosed methods include columns that utilize one or more of the following functional groups: carbamoyl groups, sulfopropyl groups, sulfoethyl groups (e.g., poly (2-sulfoethyl aspartamide)), hydroxyethyl groups (e.g., poly (2-hydroxyethyl aspartamide)) and aromatic sulfonic acid groups. Preferred functional groups include sulfoethyl groups such as poly (2-sulfoethyl aspartamide) and sulfopropyl groups such as CH₂N(CH₃)₂CH₂CH₂CH₂SO₃.

The mobile phase used includes buffers without ion pairing agents, e.g., acetonitrile (e.g., from EM Science or Burdick Jackson) and water (e.g., HPLC grade, e.g., from EM Science)). Ion paring agents include formate, acetate, and salts. Gradients of the buffers can be used, e.g., if two buffers are used, the concentration or percentage of the first buffer can decrease while the concentration or percentage of the second buffer increases over the course of the chromatography run. For example, the percentage of the first buffer can decrease from about 100%, about 99%, about 95%, about 90%, about 85%, about 80%, about 75%, about 70%, about 65%, about 60%, about 50%, about 45%, or about 40% to about 0%, about 1%, about 5%, about 10%, about 15%, about 20%, about 25%, about 30%, about 35%, or about 40% over the course of the chromatography run. As another example, the percentage of the second buffer can increase from about 0%, about 1%, about 5%, about 10%, about 15%, about 20%, about 25%, about 30%, about 35%, or about 40% to about 100%, about 99%, about 95%, about 90%, about 85%, about 80%, about 75%, about 70%, about 65%, about 60%, about 50%, about 45%, or about 40% over the course of the same run. Optionally, the concentration or percentage of the first and second buffer can return to their starting values at the end of the chromatography run. As an example, the percentage of the first buffer can change in five steps from 85% to 63% to 59% to 10% to 85%; while the percentage of the second buffer in the same steps changes from 15% to 37% to 41% to 90% to 15%. The percentages can change gradually as a linear gradient or in a non-linear (e.g., stepwise) fashion. For example, the gradient can be multiphasic, e.g., biphasic, triphasic, etc. In preferred embodiments, the methods described herein use a decreasing acetonitrile buffer gradient which corresponds to increasing polarity of the mobile phase without the use of ion pairing agents.

Because buffers free of ion pairing agents are employed, the NP-HPLC method described herein is compatible with MS, e.g., peaks isolated from HPLC can be directly analyzed without further purification (e.g., no desalting step is required). In addition, because no ion pairing agents are used, minor peaks from HPLC can be eluted, concentrated, and analyzed (e.g., off-line by MALDI) without further purification. Purification steps that are omitted include one or more of ion pairing agent removal (e.g., desalting), dialysis, drying.

The analyte, e.g., glycans, can be fluorescently labeled (derivatized), which can aid in sensitivity and/or selectivity. Fluorescence can be detected with a fluorescence detector, e.g., Waters 2475 fluorescence detector; Gilson Model 121 flow fluorometer. In other embodiments, the analyte can be radiolabeled, e.g., tritium labeled. The radiolabel can be detected using a radioactivity detector, e.g., Raytest Ramona radioisotope detector equipped with a Raytest glass scintillation flow cell. A UV detector can be used to monitor absorbance at 330 nm and 214 nm (e.g., of peptides), e.g., using a Waters 2487 dual-wavelength UV detector.

The column temperature can be maintained at a constant temperature throughout the chromatography run, e.g., using a commercial column heater. In some embodiments, the column is maintained at a temperature between about 18° C. to about 70° C., e.g., about 30° C. to about 60° C., about 40° C. to about 50° C., e.g., at about 20° C., about 25° C., about 30° C., about 35° C., about 40° C., about 45° C., about 50° C., about 55° C., about 60° C., about 65° C., or about 70° C. A preferred temperature is about 45° C.

The flow rate of the mobile phase can be between about 0 to about 100 ml/min. For analytical proposes, flow rates typically range from 0 to 10 ml/min, for preparative HPLC, flow rates in excess of 100 ml/min can be used. For example, the flow rate can be about 0.5, about 1, about 1.5, about 2, about 2.5, about 3, about 3.5, about 4, about 4.5, or about 5 ml/min. Substituting a column having the same packing, the same length, but a smaller diameter requires a reduction in the flow rate in order to retain the same retention time and resolution for peaks as seen with a column of wider diameter. Preferably, a flow rate equivalent to about 1 ml/min in a 4.6×100 mm, 5 μm column is used.

The run time for an HPLC method described herein can be between about 15 to about 240 minutes, e.g., about 20 to about 70 min, about 30 to about 60 min, about 40 to about 90 min, about 50 min to about 100 min, about 60 to about 120 min, about 50 to about 80 min. Preferably, the run time is about 60 to about 70 min, e.g., about 65 min.

The NP-HPLC can be adjusted to be performed on a nanoscale, e.g., using columns with an inner diameter of about 75 μm (see, e.g., Wuhrer et al., Anal. Chem. 76:833-838 (2004); Wuhrer et al., Internat. J. Mass. Spec. 232:51-57 (2004)).

In addition, an internal standard can be used in the methods. Suitable standards include N-glycan standards (e.g., from Prozyme) that can optionally be labeled, e.g., with 2-AB. The standards can be used to optimize the chromatographic separation and to provide a chromatographic profile for various structures (e.g., N-glycans).

The LC analyzed glycans are then further subjected to analysis by mass spectrometry. Examples of mass spectrometry that can be used to further analyze the glycans include ESI-MS, turbospray ionization mass spectrometry, nanospray ionization mass spectrometry, thermospray ionization mass spectrometry, sonic spray ionization mass spectrometry, SELDI-MS and MALDI-MS. For example, the methods described herein can be used to provide LC-evaluated glycans for on-line mass spectrometry (e.g., ESI-MS) and/or for off-line mass spectrometry (e.g., MALDI-MS) without further purification.

Electrospray Ionization-Quadrupole Time-of-Flight Mass Spectrometry (ESI-QTOF MS)

The methods described herein include coupling the LC method, e.g., the HPLC method, e.g., NP-HPLC method, online with a mass spectrometer, e.g., a quadrupole mass spectrometer, a time of flight mass spectrometer or a quadrupole time-of-flight (QTOF) mass spectrometer with electrospray ionization (ESI), e.g., Q-T of API US (Waters, Beverly, Mass.).

Ionization Source

The effluent, or at least a portion of effluent, from the LC system can be directed to the ionization source of the spectrometer. The ionization used is preferably ESI. The source can be nanospray ionization such as a Z-spray ion source. ESI is one of the atmospheric pressure ionization (API) techniques and is well-suited to the analysis of polar molecules ranging from less than 100 Da to more than 1,000,000 Da in molecular mass. Nanospray ionization (M. Wilm, M. Mann, Anal. Chem., 1996, 68, 1) is a slow flow rate version of electrospray ionization.

Generally, during standard ESI (J. Fenn, J. Phys. Chem. 88 (1984) 4451), the sample can be dissolved in a polar, volatile solvent and pumped through, e.g., a narrow, stainless steel capillary (75-150 micrometers i.d.) at a flow rate of between, e.g., 1 μL/min and 1 mL/min. A high voltage of, e.g., about 3 or 4 kV can be applied to the tip of the capillary, which is situated within the ionization source of the mass spectrometer, and as a consequence of this strong electric field, the sample emerging from the tip can be dispersed into an aerosol of highly charged droplets, a process that can be aided by a co-axially introduced nebulizing gas flowing around the outside of the capillary. This gas, e.g., nitrogen, helps to direct the spray emerging from the capillary tip towards the mass spectrometer. The charged droplets can diminish in size by solvent evaporation, assisted by a warm flow of the drying gas, e.g., nitrogen, which passes across the front of the ionization source. Eventually, charged sample ions, free from solvent, can be released from the droplets, some of which can pass through a sampling cone or orifice into an intermediate vacuum region, and from there through a small aperture into the analyzer of the mass spectrometer, which can be held under high vacuum. The lens voltages can be optimized individually for each sample.

ESI and nanospray ionization are very sensitive analytical techniques but the sensitivity deteriorates with the presence of non-volatile buffers and other additives. The methods described herein can overcome this problem.

In a positive ionization mode, a trace of formic acid can be added to aid protonation of the sample molecules; in a negative ionization mode a trace of ammonia solution or a volatile amine can be added to aid deprotonation of the sample molecules. The preferred mode of the present methods is a positive ionization mode.

The Analyzer

The main function of the mass analyzer is to separate, or resolve, the ions formed in the ionization source of the mass spectrometer according to their mass-to-charge (m/z) ratios. There are a number of mass analyzers currently available, e.g., quadrupoles, time-of-flight (TOF) analyzers, magnetic sectors, and both Fourier transform and quadrupole ion traps. The TOF analyzer uses an electric field to accelerate the ions through the same potential, and then measures the time they take to reach the detector. If the particles all have the same charge, then their kinetic energies will be identical, and their velocities will depend only on their masses. Lighter ions will reach the detector first. Quadrupole mass analyzers use oscillating electrical fields to selectively stabilize or destabilize ions passing through a radio frequency (RF) quadrupole field. A quadrupole mass analyzer acts as a mass selective filter and is closely related to the quadrupole ion trap, particularly the linear quadrupole ion trap except that is operates without trapping the ions. A common variation of the quadrupole is the triple quadrupole.

The preferred spectrometer of the present methods is a tandem (MS-MS) spectrometer that includes both a quadrupole and a TOF analyzer, or a QTOF analyzer. The two analyzers can be separated by a collision cell into which an inert gas, e.g., argon or xenon, is admitted to collide with the selected sample ions and bring about their fragmentation. The collision energy can be, e.g., 5 eV.

The Detector

The detector of the spectrometer monitors the ion current, amplifies it and transmits the signal to the data system, where it is recorded in the form of mass spectra. The m/z values of the ions are plotted against their intensities to show the number of components in the sample, the molecular mass of each component, and the relative abundance of the various components in the sample. The type of detector is supplied to suit the type of analyzer and can include the photomultiplier, the electron multiplier and the micro-channel plate detectors. In the present methods, the data can be acquired, e.g., from m/z 50 to m/z 3000, in, e.g., 2 s scans, with, e.g., 0.1 s interscan delay.

The acquired data can be analyzed with e.g., MassLynx 3.5 software (Waters, Beverly, Mass.).

MALDI-MS

Methods described herein include the use of MALDI-MS to analyze glycans from the LC effluent. In one embodiment, the MALDI ion source uses a time of flight (TOF) mass analyzer, a quadrupole mass analyzer or a quadrupole time of flight (Q-TOF). The MALDI-TOF can be linear time of flight (L-TOF) (e.g., with continuous or delayed ion extraction) or reflectron time of flight (re-TOF). Preferably, L-TOF is used with a delayed ion extraction.

Prior to MALDI-MS analysis, the LC effluent, e.g., fractions obtained by, separated by or purified by LC, is combined with a matrix. The matrix can include one or more components. For example, the matrix can include an inorganic compound, e.g., an inorganic compound having high molar absorptivity at the wavelength of the laser being used. Examples of matrices that can be used include 5-dihydroxybenzoic acid (DHB) and 2-hydroxy-5-methoxybenzoic acid (HMB).

The preparation, and/or the matrix can be deposited on the MALDI sample plate using known methods such as dried droplet method, surface preparation methods, crushed crystal methods and electrospray deposition. In some embodiments, the preparation and/or matrix is contacted with a solvent, H₂0 or a combination thereof prior to being deposited on the sample plate. Examples of solvents include acetonitrile.

Separation of ions of different m/z values in TOF mass spectrometry can be measured by the total time of flight from ion formation to impact on a detector.

### Additional Methods of Analyzing Glycomolecule Structure

The methods disclosed herein can be used in conjunction with other methods to provide information regarding glycomolecules in a preparation. For example, the methods disclosed herein can be used as part of a “top-down” or “bottom-up” analysis of a glycomolecule, e.g., a glycoprotein. “Top-down” analysis of a glycoprotein refers to analysis of the protein structure, e.g., by peptide mapping and/or peptide sequencing. “Bottom-up” analysis refers to analysis of the intact glycoprotein as well as subunits of the glycoprotein. Preferably, the methods described herein are used as part of a “bottom-up” analysis of a glycoprotein. The results of a bottom up analysis should be comparable and, e.g., complementary, to results obtained from a top-down analysis. Various methods the can be used for a top-down analysis and/or a bottom-up analysis are described herein and in J. C. Rouse, J. E. McClellan, H. K. Patel, M. A. Jankowski, T. J. Porter, “Top-Down Characterization by Liquid Chromatography/Mass Spectrometry: Application to Recombinant Factor IX Comparability-A Case Study” in: C. M. Smales, D. C. James (Eds.) Methods in Molecular Biology, vol. 308: Therapeutic Proteins: Methods and Protocols, Humana Press, Totowa, N.J., 2005, pp. 435-460.

### Glycoproteins

In certain embodiments, the glycomolecule can be a glycoprotein. Glycoproteins in the glycoprotein preparations can be produced recombinantly. The terms “recombinantly expressed glycoprotein” and “recombinant glycoprotein” as used herein refer to a glycopolypeptide expressed from a host cell that has been manipulated by the hand of man to express that glycopolypeptide. In certain embodiments, the host cell is a mammalian cell. In certain embodiments, this manipulation may comprise one or more genetic modifications. For example, the host cells may be genetically modified by the introduction of one or more heterologous genes encoding the polypeptide to be expressed. The heterologous recombinantly expressed glycopolypeptide can be identical or similar to polypeptides that are normally expressed in the host cell. The heterologous recombinantly expressed glycopolypeptide can also be foreign to the host cell, e.g., heterologous to glycopolypeptides normally expressed in the host cell. In certain embodiments, the heterologous recombinantly expressed glycopolypeptide is chimeric. For example, portions of a polypeptide may contain amino acid sequences that are identical or similar to polypeptides normally expressed in the host cell, while other portions contain amino acid sequences that are foreign to the host cell. Additionally or alternatively, a polypeptide may contain amino acid sequences from two or more different polypeptides that are both normally expressed in the host cell. Furthermore, a polypeptide may contain amino acid sequences from two or more polypeptides that are both foreign to the host cell. In some embodiments, the host cell is genetically modified by the activation or upregulation of one or more endogenous genes.

Any protein that is glycosylated can be used in accordance with the present invention. For example, the methods described herein may be employed to analyze of any pharmaceutically or commercially relevant antibody, receptor, cytokine, growth factor, enzyme, clotting factor, hormone, regulatory factor, antigen, binding agent, among others. The following list of proteins that can be analyzed according to the present invention is merely exemplary in nature, and is not intended to be a limiting recitation. One of ordinary skill in the art will understand that any glycolprotein can be evaluated and will be able to select the particular glycoprotein to be produced based as needed.

Antibodies and Binding Fragments

The methods disclosed herein can be used to evaluate or process glycan structures in a preparation of antibody or an antigen-binding fragment thereof. Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain includes an N-terminal variable (V) domain (VL) and a constant (C) domain (CL). Each heavy chain includes an N-terminal V domain (VH), three or four C domains (CHs), and a hinge region. The CH domain most proximal to VH is designated as CH1. Often, the VH domain of an antibody is glycosylated, e.g., with an N-linked glycan. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of Immunological Interest, US Department of Health and Human Services (1991), eds. Kabat et al. Another standard for characterizing the antigen binding site is to refer to the hypervariable loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Embodiments described with respect to Kabat CDRs can alternatively be implemented using similar described relationships with respect to Chothia hypervariable loops or to the AbM-defined loops. As used herein, the term “antibody” includes a protein comprising at least one, and typically two, VH domains or portions thereof, and/or at least one, and typically two, VL domains or portions thereof. In certain embodiments, the antibody is a tetramer of two heavy immunoglobulin chains and two light immunoglobulin chains, wherein the heavy and light immunoglobulin chains are inter-connected by, e.g., disulfide bonds. The antibodies, or a portion thereof, can be obtained from any origin, including, but not limited to, rodent, primate (e.g., human and non-human primate), camelid, as well as recombinantly produced, e.g., chimeric, humanized, and/or in vitro generated, as described in more detail herein.

Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain, e.g., a VHH domain; (vii) a single chain Fv (scFv); (viii) a bispecific antibody; and (ix) one or more antigen binding fragments of an immunoglobulin fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; Huston et al. (1988) Proc. Natl. Acad. Sci. U.S.A. 85:5879-83). Such single chain antibodies are also intended to be encompassed within the term “antigen-binding fragment” of an antibody. These antibody fragments are obtained using conventional techniques known to those skilled in the art, and the fragments are evaluated for function in the same manner as are intact antibodies.

Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

Numerous methods known to those skilled in the art are available for obtaining antibodies. For example, monoclonal antibodies may be produced by generation of hybridomas in accordance with known methods. Hybridomas formed in this manner are then screened using standard methods, such as enzyme-linked immunosorbent assay (ELISA) and surface plasmon resonance (Biacore™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

In another embodiment, a monoclonal antibody is obtained from the non-human animal, and then modified, e.g., humanized, deimmunized, chimeric, may be produced using recombinant DNA techniques known in the art. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982), and may be made according to the teachings of PCT Publication WO92/06193 or EP 0239400).

An antibody may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson, et al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

In certain embodiments, an antibody can contain an altered immunoglobulin constant or Fc region. For example, an antibody produced in accordance with the teachings herein may bind more strongly or with more specificity to effector molecules such as complement and/or Fc receptors, which can control several immune functions of the antibody such as effector cell activity, lysis, complement-mediated activity, antibody clearance, and antibody half-life. Typical Fc receptors that bind to an Fc region of an antibody (e.g., an IgG antibody) include, but are not limited to, receptors of the FcγRI, FcγRII, and FcγRIII and FcRn subclasses, including allelic variants and alternatively spliced forms of these receptors. Fc receptors are reviewed in Ravetch and Kinet, Annu. Rev. Immunol 9:457-92, 1991; Capel et al., Immunomethods 4:25-34, 1994; and de Haas et al., J. Lab. Clin. Med. 126:330-41, 1995).

Non-limiting examples of antibodies that can be analyzed by the methods of the invention, include but are not limited to, antibodies against IL-13, IL-22 and GDF8. Each of these antibodies is described in more detail herein below and the appended Examples.

Anti-GDF8 Antibodies

Exemplary antibodies that can analyzed by the methods disclosed herein are anti-GDF8 antibodies. The term “GDF-8” refers to growth and differentiation factor-8 and, where appropriate, factors that are structurally or functionally related to GDF-8, for example, BMP-11 and other factors belonging to the TGF-β superfamily. The term refers to the full-length unprocessed precursor form of GDF-8, as well as the mature and propeptide forms resulting from post-translational cleavage. The term also refers to any fragments and variants of GDF-8 that maintain at least some biological activities associated with mature GDF-8 including sequences that have been modified. The amino acid sequence human GDF-8, as well as many other vertebrate species (including murine, baboon, bovine, chicken) is disclosed, e.g., U.S. 04/0142382, US 02/0157125, and McPherron et al. (1997) Proc. Nat. Acad. Sci. U.S.A., 94:12457-12461). Examples of neutralizing antibodies against GDF-8, e.g., Myo-029, are disclosed in, e.g., U.S. 2004/0142382, and are referenced throughout the Examples appended herein. Exemplary disease and disorders include muscle and neuromuscular disorders such as muscular dystrophy (including Duchenne's muscular dystrophy); amyotrophic lateral sclerosis; muscle atrophy; organ atrophy; frailty; tunnel syndrome; congestive obstructive pulmonary disease; sarcopenia, cachexia, and other muscle wasting syndromes; adipose tissue disorders (e.g., obesity); type 2 diabetes; impaired glucose tolerance; metabolic syndromes (e.g., syndrome X); insulin resistance induced by trauma such as burns or nitrogen imbalance; and bone degenerative diseases (e.g., osteoarthritis and osteoporosis)

Anti-IL13 Antibodies

Interleukin-13 (IL-13) is a previously characterized cytokine secreted by T lymphocytes and mast cells (McKenzie et al. (1993) Proc. Natl. Acad. Sci. USA 90:3735-39; Bost et al. (1996) Immunology 87:663-41). The term “IL-13” refers to interleukin-13, including full-length unprocessed precursor form of IL-13, as well as the mature forms resulting from post-translational cleavage. The term also refers to any fragments and variants of IL-13 that maintain at least some biological activities associated with mature IL-3 including sequences that have been modified. The term “IL-13” includes human IL-13, as well as other vertebrate species. Several pending applications disclose antibodies against human and monkey IL-13, IL-13 peptides, vectors and host cells producing the same, for example, U.S. Application Publication Nos. 2006/0063228A and 2006/0073148. The contents of all of these publications are incorporated by reference herein in their entirety.

IL-13 shares several biological activities with IL-4. For example, either IL-4 or IL-13 can cause IgE isotype switching in B cells (Tomkinson et al. (2001) J. Immunol. 166:5792-5800). Additionally, increased levels of cell surface CD23 and serum CD23 (sCD23) have been reported in asthmatic patients (Sanchez-Guererro et al. (1994) Allergy 49:587-92; DiLorenzo et al. (1999) Allergy Asthma Proc. 20:119-25). In addition, either IL-4 or IL-13 can upregulate the expression of MHC class II and the low-affinity IgE receptor (CD23) on B cells and monocytes, which results in enhanced antigen presentation and regulated macrophage function (Tomkinson et al., supra). These observations suggest that IL-13 may be an important player in the development of airway eosinophilia and airway hyperresponsiveness (AHR) (Tomkinson et al., supra; Wills-Karp et al. (1998) Science 282:2258-61). Accordingly, inhibition of IL-13 can be useful in ameliorating the pathology of a number of inflammatory and/or allergic conditions, including, but not limited to, respiratory disorders, e.g., asthma; chronic obstructive pulmonary disease (COPD); other conditions involving airway inflammation, eosinophilia, fibrosis and excess mucus production, e.g., cystic fibrosis and pulmonary fibrosis; atopic disorders, e.g., atopic dermatitis, urticaria, eczema, allergic rhinitis; inflammatory and/or autoimmune conditions of, the skin (e.g., atopic dermatitis), gastrointestinal organs (e.g., inflammatory bowel diseases (IBD), such as ulcerative colitis and/or Crohn's disease), liver (e.g., cirrhosis, hepatocellular carcinoma); scleroderma; tumors or cancers (e.g., soft tissue or solid tumors), such as leukemia, glioblastoma, and lymphoma, e.g., Hodgkin's lymphoma; viral infections (e.g., from HTLV-1); fibrosis of other organs, e.g., fibrosis of the liver, (e.g., fibrosis caused by a hepatitis B and/or C virus).

Anti-IL22 Antibodies

Interleukin-22 (IL-22) is a previously characterized class II cytokine that shows sequence homology to IL-10. Its expression is up-regulated in T cells by IL-9 or ConA (Dumoutier L. et al. (2000) Proc Natl Acad Sci USA 97(18):10144-9). Studies have shown that expression of IL-22 mRNA is induced in vivo in response to LPS administration, and that IL-22 modulates parameters indicative of an acute phase response (Dumoutier L. et al. (2000) supra; Pittman D. et al. (2001) Genes and Immunity 2:172), and that a reduction of IL-22 activity by using a neutralizing anti-IL-22 antibody ameliorates inflammatory symptoms in a mouse collagen-induced arthritis (CIA) model. Thus, IL-22 antagonists, e.g., neutralizing anti-IL-22 antibodies and fragments thereof, can be used to induce immune suppression in vivo, for examples, for treating autoimmune disorders (e.g., arthritic disorders such as rheumatoid arthritis); respiratory disorders (e.g., asthma, chronic obstructive pulmonary disease (COPD)); inflammatory conditions of, e.g., the skin (e.g., psoriasis), cardiovascular system (e.g., atherosclerosis), nervous system (e.g., Alzheimer's disease), kidneys (e.g., nephritis), liver (e.g., hepatitis) and pancreas (e.g., pancreatitis).

The term “IL-22” refers to interleukin-22, including full-length unprocessed precursor form of IL-22, as well as the mature forms resulting from post-translational cleavage. The term also refers to any fragments and variants of IL-22 that maintain at least some biological activities associated with mature IL-22, including sequences that have been modified. The term “IL-22” includes human IL-22, as well as other vertebrate species. The amino acid and nucleotide sequences of human and rodent IL-22, as well as antibodies against IL-22 are disclosed in, for example, U.S. Application Publication Nos. 2005-0042220 and 2005-0158760, and U.S. Pat. No. 6,939,545. The contents of all of these publications are incorporated by reference herein in their entirety.

Soluble Receptors and Receptor Fusions

The invention can also be applied to soluble receptors or fragments thereof. Examples of soluble receptors include the extracellular domain of interleukin-21 receptor (IL-21R) as described in, for example, US 2003-0108549 (the contents of which are also incorporated by reference).

The fusion protein can include a targeting moiety, e.g., a soluble receptor fragment or a ligand, and an immunoglobulin chain, an Fc fragment, a heavy chain constant regions of the various isotypes, including: IgG1, IgG2, IgG3, IgG4, IgM, IgA1, IgA2, IgD, and IgE). For example, the fusion protein can include the extracellular domain of a receptor, and, e.g., fused to, a human immunoglobulin Fc chain (e.g., human IgG, e.g., human IgG1 or human IgG4, or a mutated form thereof). In one embodiment, the human Fc sequence has been mutated at one or more amino acids, e.g., mutated at residues 254 and 257 from the wild type sequence to reduce Fc receptor binding. The fusion proteins may additionally include a linker sequence joining the first moiety to the second moiety, e.g., the immunoglobulin fragment. For example, the fusion protein can include a peptide linker, e.g., a peptide linker of about 4 to 20, more preferably, 5 to 10, amino acids in length; the peptide linker is 8 amino acids in length. For example, the fusion protein can include a peptide linker having the formula (Ser-Gly-Gly-Gly-Gly)y wherein y is 1, 2, 3, 4, 5, 6, 7, or 8. In other embodiments, additional amino acid sequences can be added to the N- or C-terminus of the fusion protein to facilitate expression, steric flexibility, detection and/or isolation or purification.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that encode a fusion moiety (e.g., an Fc region of an immunoglobulin heavy chain). Immunoglobulin fusion polypeptides are known in the art and are described in e.g., U.S. Pat. Nos. 5,516,964; 5,225,538; 5,428,130; 5,514,582; 5,714,147; and 5,455,165.

### Growth Factors and Cytokines

Another class of polypeptides that can be analyzed for glycan structure includes growth factors and other signaling molecules, such as cytokines.

Growth factors are typically glycoproteins that are secreted by cells and bind to and activate receptors on other cells, initiating a metabolic or developmental change in the receptor cell. Non-limiting examples of mammalian growth factors and other signaling molecules include cytokines; epidermal growth factor (EGF); platelet-derived growth factor (PDGF); fibroblast growth factors (FGFs) such as aFGF and bFGF; transforming growth factors (TGFs) such as TGF-alpha and TGF-beta, including TGF-beta 1, TGF-beta 2, TGF-beta 3, TGF-beta 4, or TGF-beta 5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (TLs), e.g., IL-1 to IL-13 (e.g., IL-11); tumor necrosis factor (TNF) alpha and beta; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin, hemopoietic growth factor; enkephalinase; RANTES (regulated on activation normally T-cell expressed and secreted); human macrophage inflammatory protein (MIP-1-alpha); mullerian-inhibiting substance; relaxin A-chain; relaxin B-chain; prorelaxin; mouse gonadotropin-associated peptide; neurotrophic factors such as bone-derived neurotrophic factor (BDNF), neurotrophin-3, -4, -5, or -6 (NT-3, NT-4, NT-5, or NT-6), or a nerve growth factor such as NGF-beta. One of ordinary skill in the art will be aware of other growth factors or signaling molecules that can be expressed in accordance with methods and compositions of the present invention.

Specific alterations in the glycosylation pattern of growth factors or other signaling molecules have been shown to have dramatic effects on their therapeutic properties. As one example, a common method of treatment for patients who suffer from chronic anemia is to provide them with frequent injections of recombinant human erythropopietin (rHuEPO) in order to boost their production of red blood cells. An analog of rHuEPO, darbepoetin alfa (Aranesp®), has been developed to have a longer duration than normal rHuEPO. The primary difference between darbepoetin alfa and rHuEPO is the presence of two extra sialic-acid-containing N-linked oligosaccharide chains. Production of darbepoetin alfa has been accomplished using in vitro glycoengineering (see Elliott et al., Nature Biotechnology 21(4):414-21, 2003, incorporated herein by reference in its entirety). Elliott et al. used in vitro mutagenesis to incorporate extra glycosylation sites into the rHuEPO polypeptide backbone, resulting in expression of the darbepoetin alfa analog. The extra oligosaccharide chains are located distal to the EPO receptor binding site and apparently do not interfere with receptor binding. However, darbepoetin alfa's half-life is up to three-fold higher than rHuEPO, resulting in a much more effective therapeutic agent. Thus, methods of determining glycan structures, e.g., between glycoproteins such as cytokines and growth factors produced, e.g., by alternative processes or expression systems, can be useful to evaluate potential differences in activity.

Clotting Factors

Clotting factors can also be evaluated for glycan structures associated with such glycoproteins. Hemophilia B is a disorder in which the blood of the sufferer is unable to clot. Thus, any small wound that results in bleeding is potentially a life-threatening event. For example, Coagulation Factor IX (Factor 1× or “FIX”) is a single-chain glycoprotein whose deficiency results in Hemophilia B. FIX is synthesized as a single chain zymogen that can be activated to a two-chain serine protease (Factor IXa) by release of an activation peptide. The catalytic domain of Factor IXa is located in the heavy chain (see Chang et al., J. Clin. Invest., 100:4, 1997, incorporated herein by reference in its entirety). FIX has multiple glycosylation sites including both N-linked and O-linked carbohydrates. One particular O-linked structure at Serine 61 (Sia-α2,3-Gal-β1,4-GlcNAc-β1,3-Fuc-α1-O-Ser) was once thought unique to FIX but has since found on a few other molecules including the Notch protein in mammals and Drosophila (Maloney et al., Journal of Biol. Chem., 275(13), 2000). FIX produced by Chinese Hamster Ovary (“CHO”) cells in cell culture exhibits some variability in the Serine 61 oligosaccharide chain. These different glycoforms, and other potential glycoforms, may have different abilities to induce clotting when administered to humans or animals and/or may have different stabilities in the blood, resulting in less effective clotting. Hemophilia A, which is clinically indistinguishable from Hemophilia B, is caused by a defect in human clotting factor VIII, another glycoprotein that is synthesized as a single chain and then processed into a two-chain active form. The present invention may used to evaluate glycan structures associated with various preparations to determine, e.g., effect of glycan structures in the preparation on clotting activity. Other clotting factors that can be analyzed by the methods described herein include tissue factor and von Willebrands factor.

Enzymes

Another class of polypeptides that can be analyzed for glycan structure according to the invention includes enzymes. Enzymes may be glycoproteins whose glycosylation pattern affects enzymatic activity. Thus, the present invention may also be used to analyze enzymes produced in a cell culture, e.g., under different cell culture conditions and/or expression systems, to provide enzymes have a more extensive or otherwise more desirable glycosylation pattern.

As but one non-limiting example, a deficiency in glucocerebrosidase (GCR) results in a condition known as Gaucher's disease, which is caused by an accumulation of glucocerebrosidase in lysosomes of certain cells. Subjects with Gaucher's disease exhibit a range of symptoms including splenomegaly, hepatomegaly, skeletal disorder, thrombocytopenia and anemia. Friedman and Hayes showed that recombinant GCR (rGCR) containing a single substitution in the primary amino acid sequence exhibited an altered glycosylation pattern, specifically an increase in fucose and N-acetyl glucosamine residues compared to naturally occurring GCR (see U.S. Pat. No. 5,549,892).

Friedman and Hayes also demonstrated that this rGCR exhibited improved pharmacokinetic properties compared to naturally occurring rGCR. For example, approximately twice as much rGCR targeted liver Kupffer cells than did naturally occurring GCR. Although the primary amino acid sequences of the two proteins differed at a single residue, Friedman and Hayes hypothesized that the altered glycosylation pattern of rGCR may also influence the targeting to Kupffer cells. One of ordinary skill in the art will be aware of other known examples of enzymes that exhibit altered enzymatic, pharmacokinetic and/or pharmacodynamic properties resulting from an alteration in their glycosylation patterns.

### Glycoprotein Production

The invention includes methods of evaluating a glycoprotein preparation by comparing a value for a glycan structure or structures made by a first process to a value for a glycan structure or structures produced by a second process. Production of the glycoprotein preparations can vary, e.g., based on culture conditions (e.g., temperature, media, plating techniques), isolation techniques, and expression systems (e.g., recombinant versus native expression, or recombinant expression from different types of host cells, e.g., CHO versus COS cell expression).

Recombinant methods of producing glycoproteins are known in the art. Nucleotide sequences encoding the proteins are typically inserted in an expression vector for introduction into host cells that may be used to produce the desired quantity of glycopolypeptides. The term “vector” includes a nucleic acid construct often including a nucleic acid, e.g., a gene, and further including minimal elements necessary for nucleic acid replication, transcription, stability and/or protein expression or secretion from a host cell. Such constructs may exist as extrachromosomal elements or may be integrated into the genome of a host cell.

The term “expression vector” includes a specific type of vector wherein the nucleic acid construct is optimized for the high-level expression of a desired protein product. Expression vectors often have transcriptional regulatory agents, such as promoter and enhancer elements, optimized for high-levels of transcription in specific cell types and/or optimized such that expression is constitutive based upon the use of a specific inducing agent. Expression vectors further have sequences that provide for proper and/or enhanced translation of the protein As known to those skilled in the art, such vectors may easily be selected from the group consisting of plasmids, phages, viruses, and retroviruses. The term “expression cassette” includes a nucleic acid construct containing a gene and having elements in addition to the gene that allow for proper and or enhanced expression of that gene in a host cell. For producing antibodies, nucleic acids encoding light and heavy chains can be inserted into expression vectors. Such sequences can be present in the same nucleic acid molecule (e.g., the same expression vector) or alternatively, can be expressed from separate nucleic acid molecules (e.g., separate expression vectors).

The term “operably linked” includes a juxtaposition wherein the components are in a relationship permitting them to function in their intended manner (e.g., functionally linked). As an example, a promoter/enhancer operably linked to a polynucleotide of interest is ligated to said polynucleotide such that expression of the polynucleotide of interest is achieved under conditions which activate expression directed by the promoter/enhancer.

Expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences (see, e.g., Itakura et al., U.S. Pat. No. 4,704,362). In addition to the immunoglobulin DNA cassette sequences, insert sequences, and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Once the vector has been incorporated into the appropriate host cell, the host cell is maintained under conditions suitable for high level expression of the nucleotide sequences, and the collection and purification of the desired antibodies. Various host cells can be utilized to produce a glycoprotein, and using methods disclosed herein the glycan structures associated with expression in different host cells can be evaluated. In certain embodiments, the host cell is mammalian. Non-limiting examples of mammalian cells that may be used in accordance with the present invention include BALB/c mouse myeloma line (NSO/1, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); baby hamster kidney cells (BHK, ATCC CCL 10); Chinese hamster ovary cells +/−DBFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68, 1982); MRC 5 cells; FS4 cells; and a human hepatoma line (Hep G2).

Additionally, any number of commercially and non-commercially available hybridoma cell lines that express polypeptides or proteins may be used to produce a glycoprotein preparation. One skilled in the art will appreciate that hybridoma cell lines might have different nutrition requirements and/or might require different culture conditions for optimal growth and polypeptide or protein expression, and will be able to modify conditions as needed. In addition, using the methods disclosed herein, the effect, if any, of varying culture conditions can be evaluated to determine if the conditions produce varying glycan structures and/or varying levels of glycan structures.

Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. (See, e.g., Co et al., (1992) J. Immunol. 148:1149). Preferred regulatory sequences for mammalian host cell expression include viral elements that direct high levels of protein expression in mammalian cells, such as promoters and/or enhancers derived from FF-1a promoter and BGH poly A, cytomegalovirus (CMV) (such as the CMV promoter/enhancer), Simian Virus 40 (SV40) (such as the SV40 promoter/enhancer), adenovirus (e.g., the adenovirus major late promoter (AdMLP)), and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al. In exemplary embodiments, the antibody heavy and light chain genes are operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. In exemplary embodiments, the construct include an internal ribosome entry site (IRES) to provide relatively high levels of polypeptides of the invention in eukaryotic host cells. Compatible IRES sequences are disclosed in U.S. Pat. No. 6,193,980 that is also incorporated herein.

Alternatively, coding sequences can be incorporated in a transgene for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Prokaryotic host cells may also be suitable for producing the antibodies of the invention. E. coli is one prokaryotic host particularly useful for cloning the polynucleotides (e.g., DNA sequences) of the present invention. Other microbial hosts suitable for use include bacilli, such as Bacillus subtilis, enterobacteriaceae, such as Escherichia, Salmonella, and Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Expression of proteins in prokaryotes is most often carried out in E. coli with vectors containing constitutive or inducible promoters directing the expression of either fusion or non-fusion proteins. Fusion vectors add a number of amino acids to an antibody encoded therein, often to the constant region of the recombinant antibody, without affecting specificity or antigen recognition of the antibody. Addition of the amino acids of the fusion peptide can add additional function to the antibody, for example as a marker (e.g., epitope tag such as myc or flag).

Other microbes, such as yeast, are also useful for expression. Saccharomyces is a preferred yeast host, with suitable vectors having expression control sequences (e.g., promoters), an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally, Sambrook et al., Molecular Cloning: A Laboratory Manual (Cold Spring Harbor Press, 2nd ed., 1989), incorporated by reference herein in its entirety for all purposes). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

When heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms of the present invention can be separated as described herein and/or further purified according to procedures known in the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

### Glycoprotein Purification

The methods of the invention can be used to evaluate the effect, if any, of varying isolation or purification conditions on glycan structure of a glycoprotein preparation. In certain embodiments, an expressed protein is secreted into the medium and thus cells and other solids may be removed, as by centrifugation or filtering for example, as a first step in the purification process.

In some embodiments, an expressed protein is bound to the surface of the host cell. In such embodiments, the media is removed and the host cells expressing the polypeptide or protein are lysed as a first step in the purification process. Lysis of mammalian host cells can be achieved by any number of means known to those of ordinary skill in the art, including physical disruption by glass beads and exposure to high pH conditions.

A protein may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purfication: Methods in Enzymology (Methods in Enzymology Series, Vol 182), Academic Press, 1997, each of which is incorporated herein by reference in its entirety). For immunoaffinity chromatography in particular, the protein may be isolated by binding it to an affinity column comprising antibodies that were raised against that protein and were affixed to a stationary support. Affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the polypeptide or protein during the purification process. Protease inhibitors are particularly advantageous when cells must be lysed in order to isolate and purify the expressed polypeptide or protein.

One of ordinary skill in the art will appreciate that the exact purification technique may vary depending on the character of the polypeptide or protein to be purified, the character of the cells from which the polypeptide or protein is expressed, and/or the composition of the medium in which the cells were grown.

### EXAMPLES

### Example 1

### Materials

Recombinant mAbs expressed in Chinese hamster ovary (CHO) cell lines were produced and purified at Wyeth BioPharma (Andover, Mass.). The 2-aminobenzoic acid (2AB) derivatized N-glycan standards and recombinant Peptide N-glycosidase F (PNGase F) were supplied by Prozyme (San Leandro, Calif.). Native, non-recombinant (referred to as “native” PNGase F was purchased from New England Biolabs (Beverly, Mass.). Sodium iodide and 2AB were obtained from Aldrich (Milwaukee, Wis.). Sodium cyanoborohydride, glacial acetic acid, dimethyl sulfoxide and ammonium formate were purchased from Sigma (St. Louis, Mo.). The NP-HPLC (normal phase-HPLC) mobile phases consisted of HPLC grade water from EM Science (Gibbstown, N.J.) and acetonitrile from EM Science or Burdick Jackson (Muskegon, Mich.). The MALDI matrices 5-dihydroxybenzoic acid (DHB) and 2-hydroxy-5-methoxybenzoic acid (HMB) used to make the super DHB matrix were obtained from Aldrich (Milwaukee, Wis.).

### Example 2

### Preparation of Maldi Matrix

In separate vials, stock solutions of DHB and HMB were prepared by adding 500 μL of 66% acetonitrile to 6 mg of DHB and HMB. Each stock solution was vortexed for 1 minute. The super DHB matrix comprised a 9:1 (v/v) mixture of the DHB and HMB stock solutions and was vortexed for 1 minute.

### Example 3

### Release and Derivatization of N-Glycans

The recombinant mAb samples were incubated with PNGase F overnight (typically 16 hours) at 37° C. to release N-glycans. For each sample, an aliquot containing 300 μg of mAb was mixed with 2 μL native or recombinant PNGase F, 3 μL G7 buffer (New England Biolabs), and the appropriate amount of water resulting in a total volume of 30 μL. Released N-glycans were derivatized with 2AB in a manner similar to that published by Bigge et. al (J. C. Bigge, T. P. Patel, J. A. Bruce, P. N. Goulding, S. M. Charles, R. B. Parekh, Nonselective and efficient fluorescent labeling of glycans using 2-aminobenzamide and anthranilic acid, Anal. Biochem. 230 (1995) 229-238). The 2AB reagent was prepared by dissolving 47 mg 2AB and 63 mg of sodium cyanoborohydride in 1 mL of glacial acetic acid/dimethyl sulfoxide (30:70, v/v). A 10 μL aliquot of 2AB reagent was added to the PNGase F reaction mixture after 16 h. The 2AB derivatization reaction proceeded for 2 h at 65° C. Then, the derivatization mixture was lyophilized using a Thermo Electron (Milford, Mass.) Speed Vac for approximately 1.5 h.

### Example 4

### Solid Phase Extraction (SPE) of 2AB Derivatized N-Glycans

Excess reagents from the N-glycan release and derivatization reactions were removed using 3 mL SupelClean NH₂ (Supelco, St. Louis, Mo.) SPE cartridges. The SPE loading, washing, and elution solutions were prepared using stock solutions of acetonitrile (A) and 250 mM ammonium formate, pH 4 (B). The loading solution was 80% A, 20% B (v/v), the wash solution was 65% A, 35% B (v/v), and the elution solution was 20% A, 80% B (v/v). The SPE vacuum manifold setting was 5 psi for all steps in the SPE process. Dried samples containing 2AB derivatized N-glycans were re-suspended in 100 μL loading solution. The SPE cartridge was conditioned with two 0.5 mL aliquots of the loading solution before the re-suspended derivatization mixture was loaded. After loading the sample, the cartridge was washed with two 1 mL aliquots of the wash solution and the derivatized glycans were eluted with three 0.6 mL aliquots of the elution solution. After lyophilzation of the eluent, the 2AB derivatized N-glycans were reconstituted with water (100 to 150 μL) prior to analysis by NP-HPLC.

### Example 5

### Normal Phase HPLC

Various 2AB derivatized biantennary complex and high mannose N-glycan standards were used to optimize chromatographic separation and LC/MS performance. The 2AB derivatized N-glycans were profiled with a Waters (Milford, Mass.) 2695 Alliance HPLC system equipped with a Waters 2475 fluorescence detector (λ_{exc}=330 nm and λₑₘᵢₛₛ=420 nm). Additionally, a Waters 2487 dual-wavelength UV detector was used to monitor the absorbance at 330 nm and 214 nm. The NP-HPLC method employed a PolyLC (Columbia, Md.) polySulfoethyl Aspartamide column (4.6×100 mm, 5 μm) and a 1 mL/min gradient with mobile phases of acetonitrile (A) and water (B) (Table 1). The total run time was 65 min and the column temperature was maintained at 45° C. Typical injection volumes were 2 to 10 μL. For higher injection volumes, the sample solution composition was adjusted to 80% acetonitrile. For some mAb samples, chromatographically separated 2AB derivatized N-glycans were collected and lyophilized prior to further structural analysis by MALDI-quadrupole time-of-flight mass spectrometer (QTOF) MS/MS.

### Example 6

### On-Line NP-HPLC/MS

On-line NP-HPLC/MS experiments were performed with a Q-T of API US (Waters, Beverly, Mass.) mass spectrometer. Data was acquired and analyzed with MassLynx 3.5 software (Waters, Beverly, Mass.). The effluent from the HPLC system was split postcolumn so that ˜100 μL/min was directed to the electrospray ionization (ESI) source of the mass spectrometer. All experiments were performed in the positive ion mode with an ESI voltage of 3.0 kV, ion source cone voltage of 25 V, and collision energy (CE) of 5 eV. Data was acquired from m/z 50 to m/z 3000 in 2 s scans with 0.1 s interscan delay. Sodium iodide was used to calibrate the instrument, and during calibration the ion source temperature was 80° C. and the desolvation temperature was 120° C. After calibration, the dynamic calibration temperature compensation circuitry was activated for LC/MS, which is performed with higher temperatures (115° C. and 275° C., respectively).

### Example 7

### MALDI MS Sample Preparation

For each MALDI experiment, the 2AB derivatized N-glycans were reconstituted with 2 to 20 μL water prior to spotting. A 2 μL aliquot of glycan solution was placed on a stainless steel target. The total amount of glycan in the 2 μL aliquot ranged from approximately 10 to 50 pmol. Once the glycan solution had dried on the target, 1.5 μL of super DHB matrix solution was placed on the surface of the dried glycans. The sample and matrix were mixed in the micropipette tip used to deposit the matrix before final dispensation onto the target. The sample spot was allowed to air dry prior to analysis. The super DHB matrix provided a uniform sample/matrix surface that facilitated reproducible fragment ion yields.

### Example 8

### MALDI-QTOF MS/MS

Off-line MS/MS structural analyses of 2AB derivatized N-glycan standards and mAb N-glycan fractions collected from the NP-HPLC/MS method were performed with a MALDI Q-T of Premier (Waters, Beverly, Mass.) mass spectrometer. This instrument was equipped with a 337 nm nitrogen laser and the laser fluence ranged from 300 to 325. The [M+Na]⁺ precursor ions were isolated using approximately a 5 Da window. The CE was optimized to produce, to the extent possible, a balanced distribution of product ion abundances ranging from m/z 200 to the precursor ion m/z. Argon was the collision gas. The laser spot was incrementally maneuvered back and forth across the sample/matrix in a consistent manner as to maintain constant signal levels. Each MS/MS spectrum shown herein is the average of at least 60 scans.

### Example 9

### Analysis of Glycans from mAbs from CHO Cell Expression

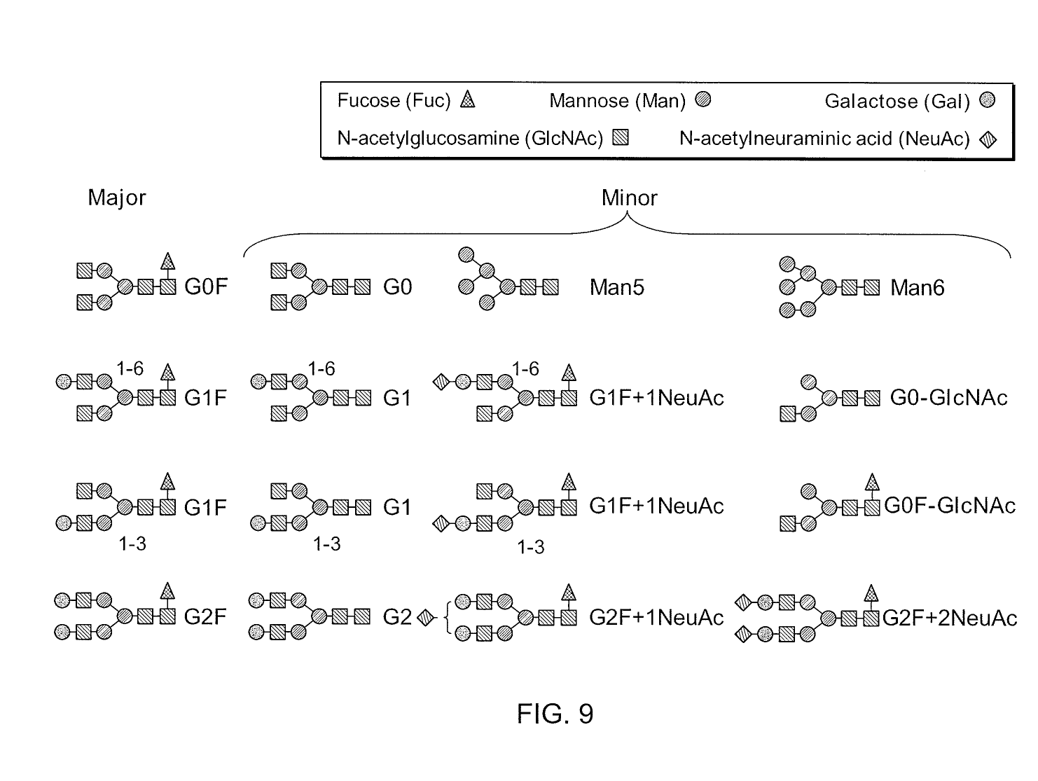

The N-glycans commonly observed for mAbs derived from CHO cell expression systems are shown in FIG. 9. The N-glycosylation of a mAb is dependent on the type of host cell line (T. S. Raju, J. B. Briggs, S. M. Borge, A. J. S. Jones, Species-specific variation in glycosylation of IgG: Evidence for the species-specific sialylation and branch-specific galactosylation and importance for engineering recombinant glycoprotein therapeutics, Glycobiology, 10 (2000) 477-486) as well as cell culture conditions (N. Jenkins, R. B. Parekh, D. C. James, Getting the glycosylation right: Implications for the biotechnology industry, Nat. Biotechnol. 14 (1996) 975-981; A. E. Hills, A. Patel, P. Boyd, D. C. James, Metabolic control of recombinant monoclonal antibody N-glycosylation in GS-NS0 cells, Biotech. and Bioengineer. 75 (2001) 239-251). Typically, the most abundant N-glycans in mAb samples are complex-type, core-fucosylated bianntenary structures that differ in the number of terminal galactose residues (G0F, G1F, G2F) (R. J. Harris, S. J. Shire, C. Winter, Commercial manufacturing scale formulation and analytical characterization of therapeutic recombinant antibodies, Drug Dev. Res. 61 (2004) 137-154; R. Jefferis, Glycosylation of recombinant antibody therapeutics, Biotechnol. Prog. 21 (2005) 11-16). Minor mAb N-glycans include non-fucosylated G0, G1, and G2 in addition to sialylated versions of the major species. High mannose structures such as oligomannose 5 (Man5) or 6 (Man6) and hybrid structures, if observed, are less abundant than the complex-type biantennary carbohydrates. Thus, in the development of an on-line NP-HPLC/MS method for profiling and characterizing mAb N-glycans, the ability to detect each of these types of structures was a primary consideration.

The objectives for an on-line NP-HPLC/MS N-glycan profiling method were achieved with the combination of acetonitrile (A) and water (B) mobile phases without ion pairing reagents or salts, the gradient elution conditions specified in Table 1, and a polySulfoethyl Aspartamide column, which was originally developed for the separation of polypeptides via strong cation-exchange (SCX) HPLC [A. J. Alpert, P. C. Andrews, Cation-exchange chromatography of peptides on poly(2-sulfoethyl aspartamide)-silica, J. Chromatogr. 443 (1988) 85-96].

TABLE 1
Gradent conditions for NP-HPLC/MS method

| Time (min) | % A | % B |
|---|---|---|
| 0.01 | 85.0 | 15.0 |
| 26.00 | 63.0 | 37.0 |
| 42.00 | 59.0 | 41.0 |
| 43.00 | 10.0 | 90.0 |
| 44.00 | 10.0 | 90.0 |
| 45.00 | 85.0 | 15.0 |

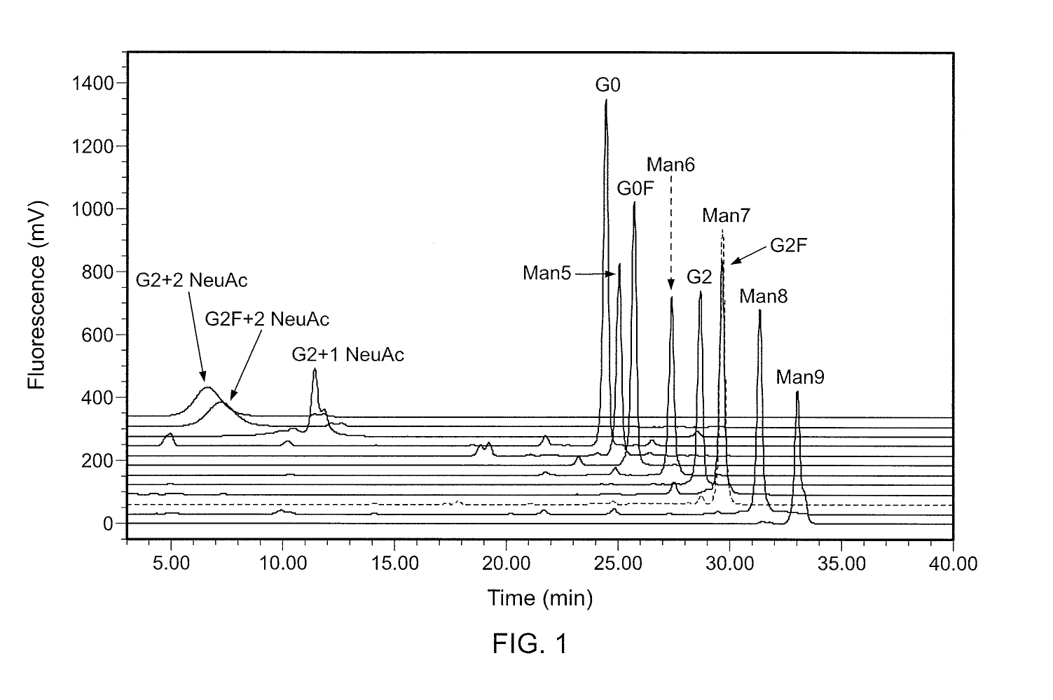

In FIG. 1, representative NP-HPLC fluorescence profiles of commercially available, 2AB-derivatized standards are shown to highlight chromatographic behavior and resolution of the N-glycan structures commonly observed in CHO-derived mAbs (Scheme 1). The sialylated N-glycans were separated chromatographically from the neutral biantennary and oligomannose carbohydrates by >10 minutes. The nonfucosylated species, G0 and G2, were baseline resolved chromatographically from the fucosylated species, G0F and G2F. The oligomannose N-glycans were separated from one another and from the complex N-glycans with the exception of possible co-elution of Man7 and G2F. However, the potential co-elution of these carbohydrates was not considered a major problem since in our experience Man7 is not commonly observed on CHO cell-derived mAbs and if observed, on-line NP-HPLC/MS can easily distinguish Man7 from G2F because they differ in mass by 228 Da.

The use of partially aqueous mobile phases and the polySulfoethyl Aspartamide column in the NP-HPLC method may be considered as hydrophilic interaction chromatography (HILIC), which is an established separation technique for polypeptides, carbohydrates, and nucleic acids (A. J. Alpert, Hydrophilic-interaction chromatography for the separation of peptides, nucleic acids and other polar compounds, J. Chromatogr. 499 (1990) 177-196; B-Y. Zhu, C. T. Mant, R. S. Hodges, Hydrophilic-interaction chromatography of peptides on hydrophilic and strong cation-exchange columns, J. Chromatogr. 548 (1991) 13-24; B-Y. Zhu, C. T. Mant, R. S. Hodges, Mixed-mode hydrophilic and ionic interaction chromatography rivals reversed-phase liquid chromatography for the separation of peptides, J. Chromatogr. 594 (1992) 75-86; T. Yoshida, Peptide separation by hydrophilic-interaction chromatography: a review, J. Biochem. Biophys. Methods 60 (2004) 265-280). HILIC is a mode of separation based on the polar interactions between analytes in the eluent and a stationary phase that is enriched with a water layer, where retention increases proportionally with the analyte hydrophilicity and decreases with the polarity of the semi-aqueous eluents, assuming limited secondary ionic interactions (A. J. Alpert, Hydrophilic-interaction chromatography for the separation of peptides, nucleic acids and other polar compounds, J. Chromatogr. 499 (1990) 177-196). In HILIC mode, polySulfoethyl Aspartamide columns have been used primarily for polypeptide separations using aqueous acetonitrile mobile phases with either a salt gradient or an acetonitrile:water gradient in which both mobile phases contain an ion pairing reagent. The NP-HPLC method described here with the polySulfoethyl Aspartamide column utilizes a decreasing acetonitrile gradient via water, without salts or ion pairing reagents, which corresponds to an increase of polarity in the mobile phase. The baseline separation of the neutral, asialo biantennary oligosaccharides, G0F and G2F, and the oligomannose species in FIG. 1 demonstrated that charge is not necessary in the retention mechanism. However, some ionic interactions are still observed, based on the observation that the negatively charged, sialylated oligosaccharides elute ˜10 minutes earlier than the neutral oligosaccharides in FIG. 1. Interestingly, when mobile phase B was replaced with 40-250 mM ammonium formate (pH 4), the sialylated N-glycans eluted ˜two minutes later than the neutral species (data not shown). Perhaps, with two mobile phases devoid of salt ions or ion pairing reagents, the sialic acid-containing N-glycans are repelled by the negatively-charged sulfoethyl resin, which promotes early elution. However, in the presence of mobile phase buffers with higher ionic strength via salts and ion pairing reagents, ionic repulsion is suppressed, which allows the more hydrophilic sialylated N-glycans to bind tighter to the water-enriched solvent layer (adsorbed onto the surface of the stationary phase) than the asialo biantennary and oligomannose N-glycans.

Four HILIC columns with unique stationary phases and two SCX columns with stationary phases similar to the polySulfoethyl Aspartamide column were surveyed for their effectiveness in the reproducible separation of mAb-derived N-glycans, overall peak shape and resolution, and recovery of the sialylated N-glycans, using the identical aqueous acetonitrile mobile phases, flow rate, temperature, and gradient elution program (Table 1), as in the NP-HPLC/MS method. The PolyHydroxyethyl Aspartamide HILIC column (2.1×100 mm) from PolyLC resulted in reproducible elution of the neutral N-glycans and unpredictable recovery of the sialylated N-glycan with the simple acetonitrile:water gradient, however full recovery of all structures was achieved reproducibly with the addition of 40 mM ammonium formate. With the Agilent Zorbax 300 SCX (4.6×150 mm, aromatic sulfonic acid moiety) and the Waters Atlantis HILIC Silica (2.1×50 mm) columns, no sialylated N-glycans were recovered using the simple acetonitrile:water gradient, but there was improved recovery through the use of 40 mM ammonium formate. The Applied Biosystems Poros® HS column (4.6×100 mm, polyhydroxylated polymer functionalized with sulfopropyl), intended for SCX-HPLC, produced broad peaks and poor resolution via the acetonitrile:water gradient with and without 40 mM ammonium formate, but sialylated N-glycans were recovered. The ZIC®-HILIC column (4.6×100 mm, sulfobetaine) from SeQuant AB [D] best mimic the separation of the polySulfoethyl Aspartamide column with the acetonitrile:water gradient in terms of sialylated N-glycan recovery and reproducible neutral N-glycan elution positions, which likely originates from the high molecular similarity of the stationary phases, however, better peak shape and resolution were obtained with the polySulfoethyl Aspartamide column. Last, the TSKgel Amide-80 column from Tosoh Bioscience (2.0×250 mm, carbamoyl) also mimicked the separation of the polySulfoethyl Aspartamide column very well, but the peak resolution was slightly less.

### Example 10

### On-Line LC/MS Profiling of 2AB mAb Derivatized N-Glycans

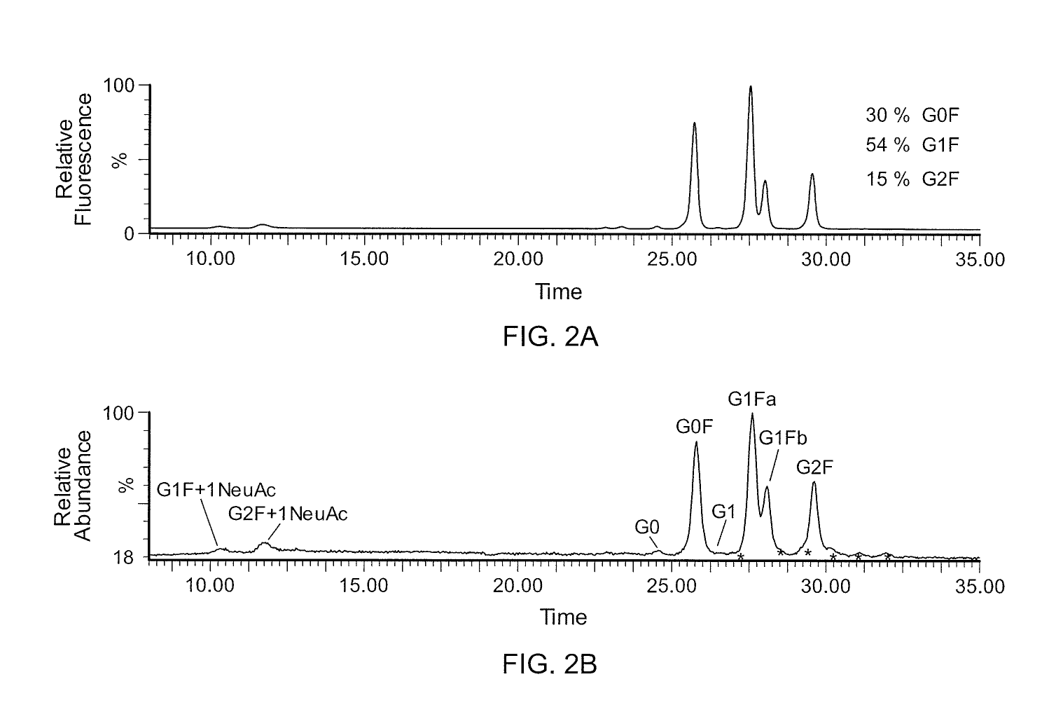

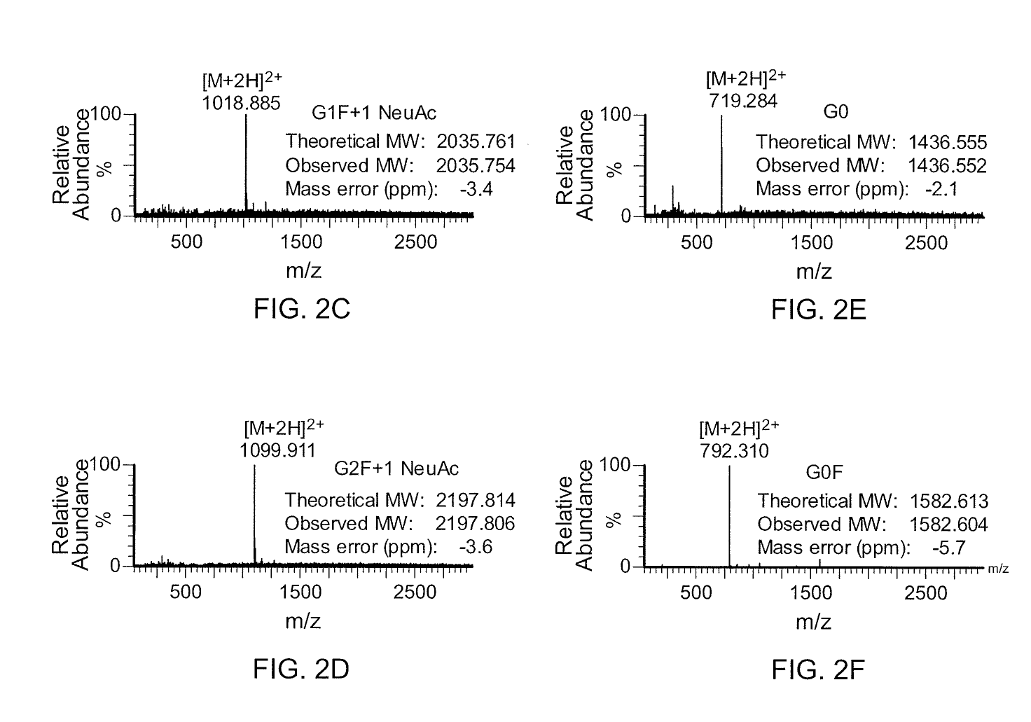

The N-glycan profiles of several IgG1 and IgG4 mAbs have been characterized using the on-line NP-HPLC/MS method. These mAbs have predominantly core-fucosylated, asialo-biantennary complex-type N-glycans with 0 to 2 terminal galactose residues (G0F, G1F, G2F) as shown in the analysis of carbohydrates released from a well-characterized IgG1 mAb (FIG. 2). The minor nonfucosylated species, G0 and G1, were chromatographically baseline resolved from the major fucosylated species, G0F and G1F, respectively. Additionally, partial chromatographic resolution of the two G1F branching isomers was achieved with this profiling method. Correlation of peaks in the fluorescence profile (FIG. 2A) and the total ion chromatogram (TIC) (FIG. 2B) provided assurance that the mass spectrometer was responding to the fluorescently labeled analyte throughout the chromatographic separation and demonstrated the compatibility of the new NP-HPLC method with MS. Predominant, doubly protonated N-glycan ions were noted in the corresponding mass spectra with minimal levels of method related artifacts (FIG. 2C to 2F). Effective concentration of signal in mainly one ionic form for each carbohydrate combined with the high mass accuracy afforded by the QTOF mass spectrometer allowed confident assignments of N-glycan identity, even for low-level species in the fluorescence profile. Thus, a primary advantage of this LC/MS method is the minimization of buffer related adducts, multiple ion types ([M+H]⁺, [M+Na]⁺) of the same oligosaccharide and ions indicative of fragmentation during LC/MS such as low m/z oxonium ions and loss of sialic acid containing antennal arms. In addition to the 2AB derivatized N-glycan ions, low-level underivatized, non-reduced and reduced G0F, G1F, and G2F ions were detected, indicating this method can be utilized to monitor the extent of 2AB derivatization.

The impact of different 2AB derivatization procedures on the N-glycan profile was examined using the on-line NP-HPLC/MS method. The relative abundance of underivatized species detected in the TIC and corresponding mass spectra increased when the mixture of mAb, released N-glycans, and PNGase F was lyophilized prior to the addition of the 2AB solution. Trace levels of underivatized carbohydrates were noted in samples where the 2AB solution was directly added to the mAb, N-glycan, and PNGase mixture that had not been lyophilized. Similar fluorescence and MS profiles were noted regardless of whether the sample was derivatized with freshly prepared 2AB solution or one that was several weeks old. Furthermore, reproducible profiles from the on-line NP-HPLC/MS method were observed intraday and day-to-day. However, the chromatographic resolution decreased and the retention time shifted after ˜20 injections. Under these circumstances, sodium adducts and 2AB adducts were observed in the mass spectra of the glycans. The presence of these adducts indicated that excess salts were accumulating on the column. After two blank injections with 40 mM ammonium formate instead of water as mobile phase B followed by two blank injections with water as mobile phase B, the performance of the LC/MS method improved and the fluorescence and MS profiles again resembled those obtained initially.

To verify the results obtained with the on-line NP-HPLC/MS profiling method, the relative ratios of the major N-glycans are compared with those derived from on-line RP-HPLC/MS of the light chain and glycosylated heavy chain subunits as well as mAb tryptic glycopeptides. Since these mAb characterization approaches that analyze the N-linked oligosaccharides rather than released N-glycans, they can be viewed as complementary and the results should agree among the three methods. As an example, the relative ratios of the major N-glycans detected G0F, G1F, and G2F detected by the N-glycan profiling method (30:54:15), matched well with the distributions observed on the intact heavy chain subunit (30:54:16) and the tryptic glycopeptide (28:55:17). The consistency of results among these independent methods of analysis demonstrated that the results from the on-line NP-HPLC/MS method were not biased and validated the inclusion of the method in the “top-down” and “bottom-up” approach for characterizing mAb N-glycan heterogeneity. Likewise, similarities in the relative abundance data from chromatographic profiling of released N-glycans and on-line RP-HPLC/MS of the intact mAb (J. Siemiatkoski, Y. Lyubarskaya, D. Houde, S. Tep, R. Mhatre, A comparison of three techniques for quantitative carbohydrate analysis used in the characterization of therapeutic antibodies, Carbohydrate Res. 341 (2006) 410-419) as well as on-line RP-HPLC/MS of the light chain and glycosylated heavy chain subunits and the tryptic glycopeptide mapping (D. S. Rehder, T. M. Dillon, G. D. Pipes, P. V. Bondarenko, Reversed-phase liquid chromatography/mass spectrometry analysis of reduced monoclonal antibodies in pharmaceutics, J. Chromatogr. A 1102 (2006) 164-175) have been reported.

### Example 11

### MALDI-QTOF MS/MS of 2AB Derivatized mAb N-Glycans

Although accurate mass data provides information about carbohydrate residue composition, it does not give specific information about the glycan structures. While some structural information can be obtained by comparing the elution position of the chromatographic peaks with those of standards, MS/MS provides more definitive structural information. Fragmentation facilitates identification of the location of monosaccharides within the glycan structure (e.g. non-reducing end vs. reducing end) and branching, especially for high mannose glycans (D. J. Harvey, Matrix-assisted laser desorption/ionization mass spectrometry of carbohydrates, Mass Spectrom. Rev. 18 (1999) 349-451). Isomers can be distinguished on the basis of their MS/MS spectra (D. J. Harvey, Matrix-assisted laser desorption/ionization mass spectrometry of carbohydrates, Mass Spectrom. Rev. 18 (1999) 349-451; D. M. Sheeley, V. N. Reinhold, Structural characterization of carbohydrate sequence, linkage, and branching in a quadrupole ion trap: Neutral oligosaccharides and N-linked glycans, Anal. Chem. 70 (1998) 3053-3059.) and several groups have proposed “knowledge-based” strategies for identifying glycans based on comparisons of MS/MS spectra of unknowns and MS/MS spectral libraries of standards (J. C. Rouse, A. M. Strang, W. Yu, J. E. Vath, Isomeric differentiation of asparagine-linked oligosaccharides by matrix-assisted laser desorption-ionization postsource decay time-of-flight mass spectrometry, Anal. Biochem. 256 (1998) 33-46; Y. Takegawa, S. Ito, S. Yoshioka, K. Deguchi, H. Nakagawa, K. Monde, S.-I. Nishimura, Structural assignment of isomeric 2-aminopyridine-derivatized oligosaccharides using MSⁿ spectral matching, Rapid Commun. Mass Spectrom. 18 (2004) 385-391). Here, structural information from MALDI-QTOF MS/MS was utilized to confirm the assignments of major and minor peaks in the NP-HPLC profiles of mAb N-glycans based on accurate mass data.

Chromatographically separated species were collected, concentrated, and analyzed directly with MALDI-QTOF MS/MS. No sample clean-up procedures were necessary prior to performing the MALDI experiments because the NP-HPLC mobile phases did not contain buffer salts. Off-line acquisition of MS/MS data with a MALDI-QTOF mass spectrometer provided several advantages. Predominantly [M+Na]⁺ glycan ions were formed by MALDI and previous studies demonstrated that these ions yield the most informative fragmentation spectra (D. J. Harvey, Electrospray mass spectrometry and fragmentation of N-linked carbohydrates derivatized at the reducing terminus, J. Am. Soc. Mass Spectrom. 11 (2000) 900-915). The QTOF afforded accurate masses for precursor and fragment ions and permitted reproducible, full m/z range, low energy fragmentation. The CE was optimized for each glycan precursor ion to yield a distribution of product ions ranging from m/z 200 to the precursor ion m/z. Each 2AB glycan had unique, reproducible fragmentation patterns and product ion intensities that facilitated a knowledge-based characterization approach in which MS/MS spectra from the 2AB derivatized mAb N-glycans were compared with those of commercially obtained 2AB N-glycan standards.

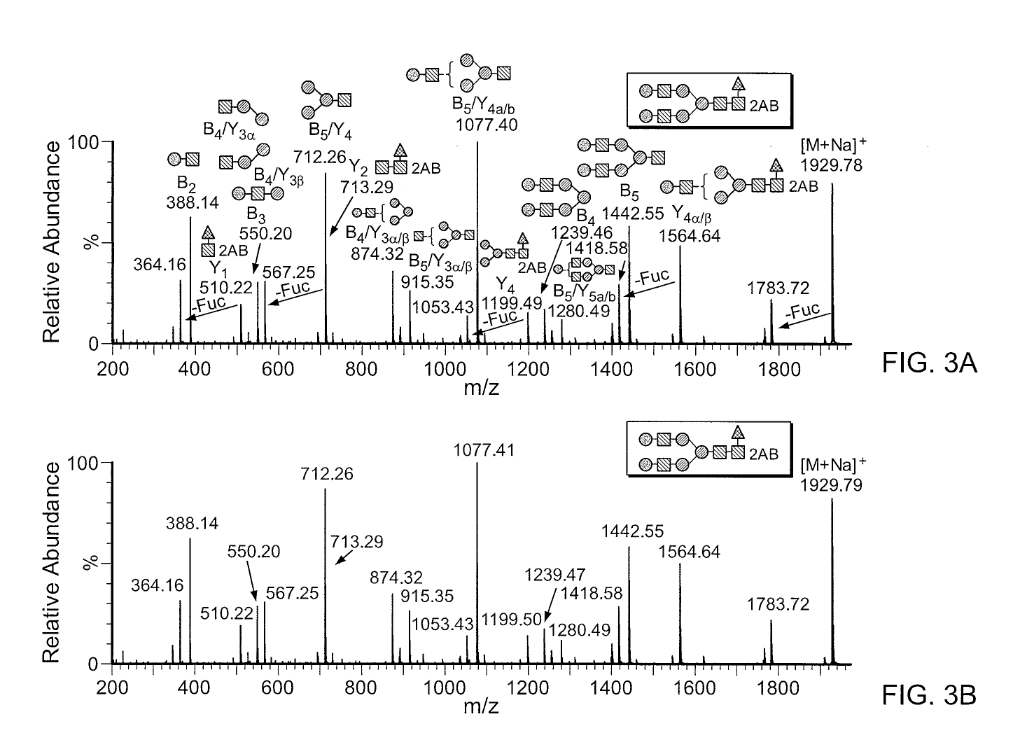

For example, a fraction containing a mAb-derived glycan that by mass corresponded to 2AB labeled G2F was collected and subjected to MALDI-QTOF MS/MS. The resulting MS/MS spectrum of [M+Na]⁺ ions from this fraction was compared to that of a commercially available 2AB labeled G2F standard (FIG. 3). The similarities between the fragment ion mass and relative abundance data in the MS/MS spectra of the standard and the collected glycan fraction verified that the fraction collected did indeed contain a carbohydrate and it had a the structure of a G2F glycan. Additionally, the spectra in FIG. 3 resembled an ESI-QTOF MS/MS spectrum of [M+Na]⁺ 2AB derivatized G2F ions published by Harvey (D. J. Harvey, Electrospray mass spectrometry and collision-induced fragmentation of 2-aminobenzamide-labelled neutral N-linked glycans, Analyst 125 (2000) 609-617). This served as confirmation of the reproducibility of QTOF MS/MS spectra of 2AB derivatized N-glycans and provided external validation of our MS/MS data. Although verification of the assignment of the G2F glycan in the NP-HPLC profile was described here, the identities of other major and minor glycans in the mAb N-glycan profiles were confirmed in a likewise manner.

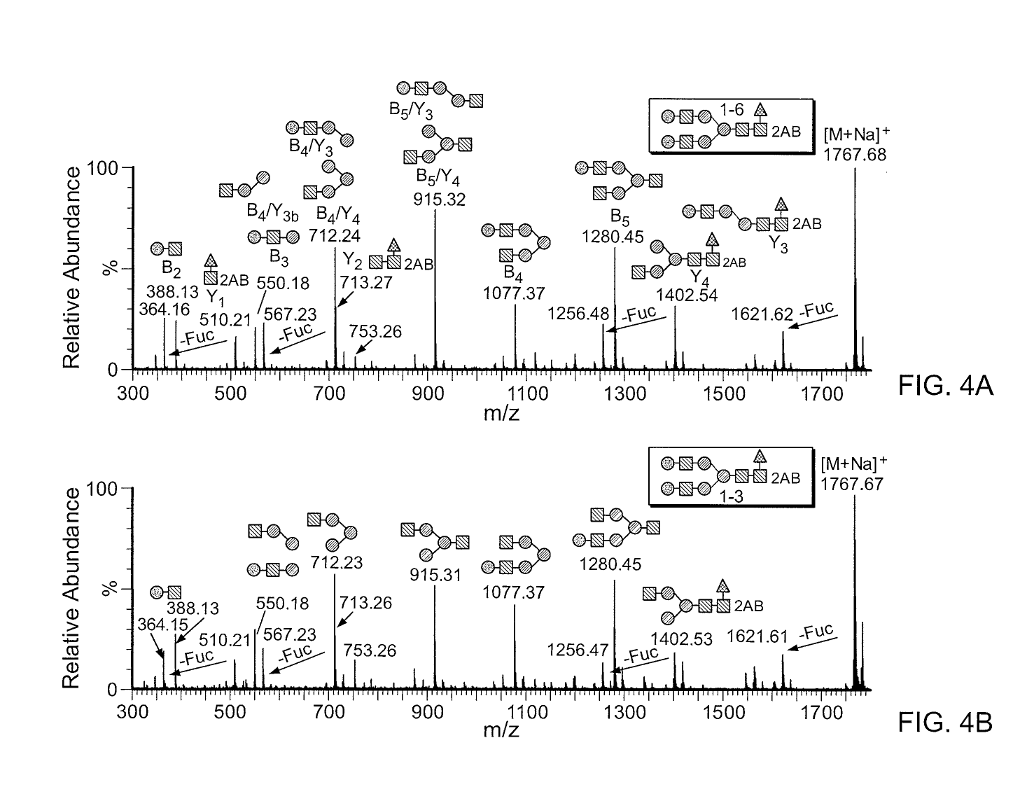

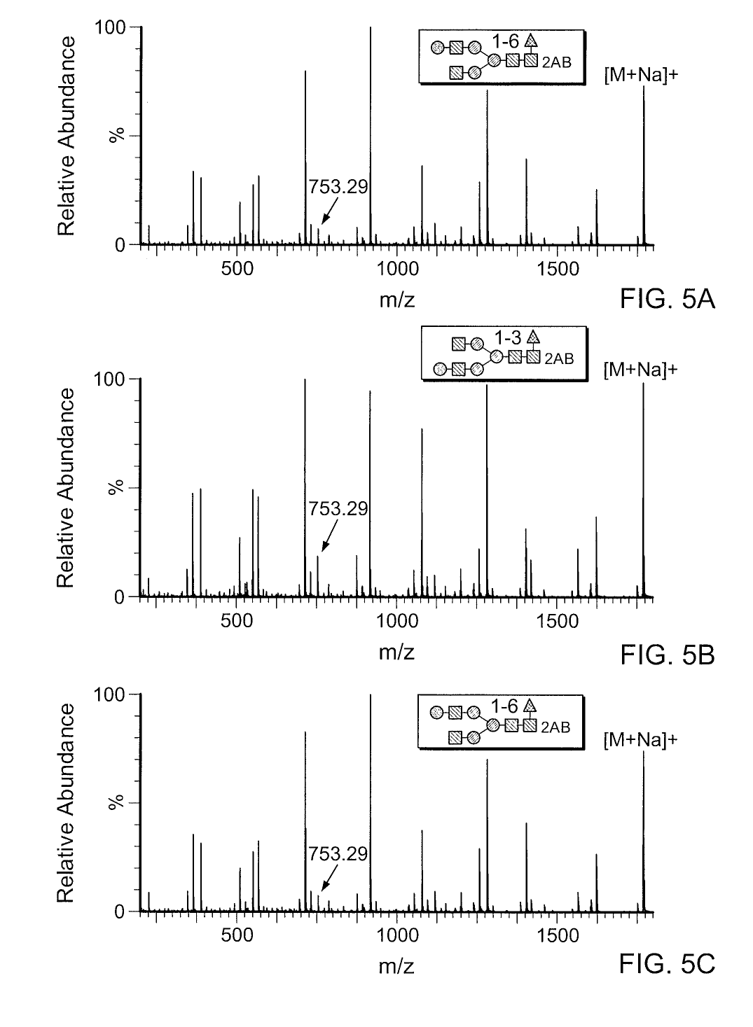

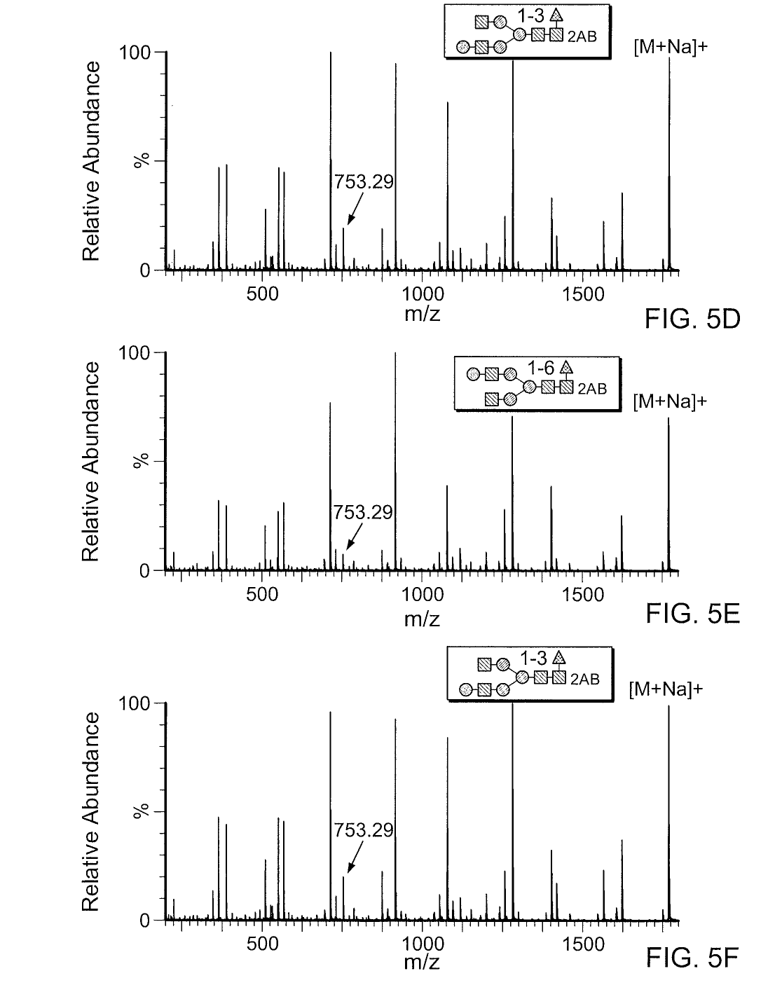

Additionally, MALDI-QTOF MS/MS was applied to differentiate the G1F isomers detected in the N-glycan profiles of IgG1 and IgG4 mAbs. These isomers differ in the location of the non-reducing terminal galactose residue on the α(1,6) or α(1,3) arm of the biantennary structure. Since G1F standards were not commercially available, chromatographic fractions of each isomer were collected initially from the N-glycan profile of an IgG4 mAb reference standard, which contained relatively high percentages of each of these isomers. The fractions were individually rechromatographed with the N-glycan profiling method, collected, lyophilized, and subjected to MALDI-QTOF MS/MS. When comparing the MS/MS spectra of [M+Na]⁺ G1Fa and G1Fa ions (FIG. 4), unique distributions of isobaric fragment ions were observed. Recently, differentiation of 2AP derivatized G1 branching isomers was achieved based on reproducible differences in relative intensity ratios of fragment ions in ion trap MS/MS and MSⁿ spectra (Y. Takegawa, S. Ito, S. Yoshioka, K. Deguchi, H. Nakagawa, K. Monde, S.-I. Nishimura, Structural assignment of isomeric 2-aminopyridine-derivatized oligosaccharides using MSⁿ spectral matching, Rapid Commun. Mass Spectrom. 18 (2004) 385-391; Y. Takegawa, K. Deguchi, S. Ito, S. Yoshioka, A. Sano, K. Yoshinari, K. Kobayashi, H. Nakagawa, K. Monde, S.-I. Nishimura, Assignment and quantification of 2-aminopyridine derivatized oligosaccharide isomers coeluted on reversed-phase HPLC/MS by MSⁿ spectral library, Anal. Chem. 76 (2004) 7294-7303; Y. Takegawa, K. Deguchi, S. Ito, S. Yoshioka, H. Nakagawa, S.-I. Nishimura, Structural assignment of isomeric 2-aminopyridine-derivatized oligosaccharides using negative-ion MSⁿ spectral matching, Rapid Commun. Mass Spectrom. 19 (2005) 937-946; N. Ojima, K. Masuda, K. Tanaka, O, Nishimura, Analysis of neutral oligosaccharides for structural characterization by matrix-assisted laser desorption/ionization quadrupole ion trap time-of-flight mass spectrometry, J. Mass Spectrom. 40 (2005) 380-388). Here, the reproducible MS/MS spectra of 2AB derivatized G1Fa and G1Fb (FIG. 5) from different mAbs suggested that the G1F isomers could be distinguished by QTOF MS/MS. Specifically, differences in the relative abundance of the m/z 753 ion in the MS/MS spectra appeared to indicate whether the terminal galactose residue was on the α(1,6) arm or the α(1,3) arm since a relative decrease or increase in the abundance of the m/z 753 ion was consistently observed for G1F having a terminal galactose residue on the α(1,6) arm or α(1,3) arm, respectively. Thus, based on the fragmentation patterns in FIGS. 4 and 5, the G1Fa isomer was believed to be α(1,6) galactosylated and the G1Fb isomer was α(1.3) galactosylated.

### Example 12

### Investigation of Minor Peaks in the N-Glycan Profiles of an IgG1 mAb

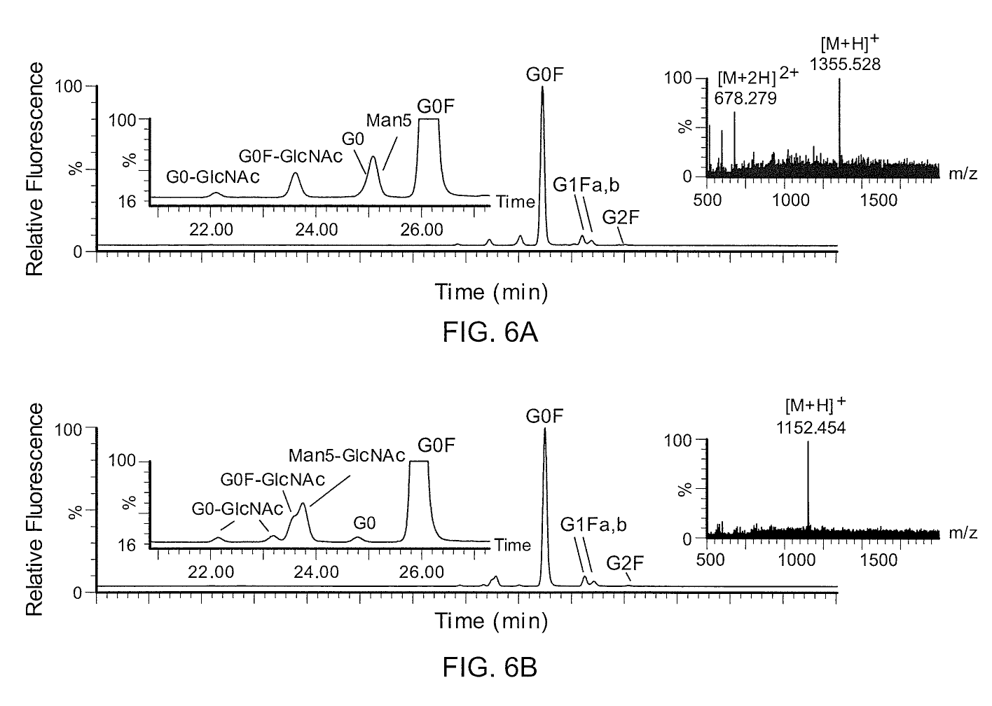

In separate experiments, N-glycans from an IgG1 reference standard were released using native and recombinant PNGase F, derivatized with 2AB, and analyzed by on-line NP-HPLC/MS. Although the relative ratios of G0F, G1F, and G2F in the NP-HPLC profiles were consistent regardless of whether the N-glycans were released with native or recombinant PNGase F, variability in minor peaks eluting before G0F was observed (FIG. 6). Accurate mass data from the on-line NP-HPLC/MS experiments and structurally informative fragment ions from MALDI-QTOF MS/MS data were used to identify the carbohydrates represented by these minor peaks. As examples, the identification of Man₅GlcNAc₂ (Man5) related carbohydrates is discussed.

While Man5 is observed when the mAb N-glycans were released with recombinant PNGase F, there is no indication of Man5 in the profiles of N-glycans released with native PNGase F (FIG. 6). Instead, a peak nominally corresponding to Man5 with only one GlcNAc residue (Man5-GlcNAc) is noted in the profile of carbohydrates released with native PNGase F (FIG. 6B). The mass spectra of the low-level Man5 related peaks from the profiles of N-glycans released with recombinant PNGase F and native PNGase F are shown as insets (FIGS. 6A and 6B). These lower mass oligomannose structures (<1400 Da) are primarily singly protonated rather than doubly protonated.

For the glycan released by recombinant PNGase F, good agreement between the observed mass, 1354.520 Da, and the theoretical mass, 1354.502 Da, of Man5 indicates that the Man5 is a carbohydrate of this mAb sample. For the glycan released by native PNGase F, accurate mass data was used to determine the carbohydrate composition of the glycan with an observed mass of 1151.446 Da (Table 2).

TABLE 2
Possible carbohydrate compositions for the 2AB derivatized
1151.446-Da N-glycan based on accurate mass data

| Glycan | Δ mass | | |
|---|---|---|---|
| mass (Da)^{a} | (ppm error)^{b} | | Structure^{c} |
| 1013.264 | 0.102 | (100.7) | (HexNAc)₁ (NeuAc)₁ (NeuGc)₁ (Pentose)₁ |
| | | | (Sulfate)₁ |
| 1013.274 | 0.092 | (90.8) | (HexNAc)₁ (NeuAc)₁ (NeuGc)₁ (Pentose)₁ |
| | | | (Phosphate)₁ |
| 1013.301 | 0.065 | (64.1) | (Hexose)₂ (HexNAc)₃ (Sulfate)₁ |
| 1013.310 | 0.056 | (55.3) | (Hexose)₂ (HexNAc)₃ (Phosphate)₁ |
| 1013.343 | 0.023 | (22.7) | (Hexose)₅ (HexNAc)₁ |

^{a}The mass of 1013.366 Da would correspond to the monosaccharide residue composition of the 1151.446-Da N-glycan without 2AB derivatization and the reducing end group. The masses in this column correspond to the sum of the monosaccharide residue masses for the glycan structures listed in the Structure column of this table.
^{b}The Δ mass and ppm error were calculated relative to the underivatized glycan mass of 1013.366 Da.
^{c}Abbreviations of carbohydrate residues: N-acetylhexosamine (HexNAc) and N-glycolyhieuraminic acid (NeuGc).

The only combination of carbohydrate residues within the mass error of the experiment (30 ppm) was Hex₅HexNAc (23 ppm). This supported the tentative identification of the minor peak in the profile of native PNGase F released glycans as Man5-GlcNAc (FIG. 6B). A mass consistent with Man5, but not 1151.446 Da was noted when examining the data from on-line RP-HPLC/MS of the tryptic glycopeptides and glycosylated heavy chain of this mAb sample. These independent analyses confirmed that Man5 was a carbohydrate of this mAb sample. Because ions associated with a glycan mass of 1151.446 Da were not observed in the analysis of the tryptic glycopeptides or the glycosylated heavy chain, the oligosaccharide detected in the NP-HPLC/MS profile was likely an artifact of the N-glycan release with native PNGase F.

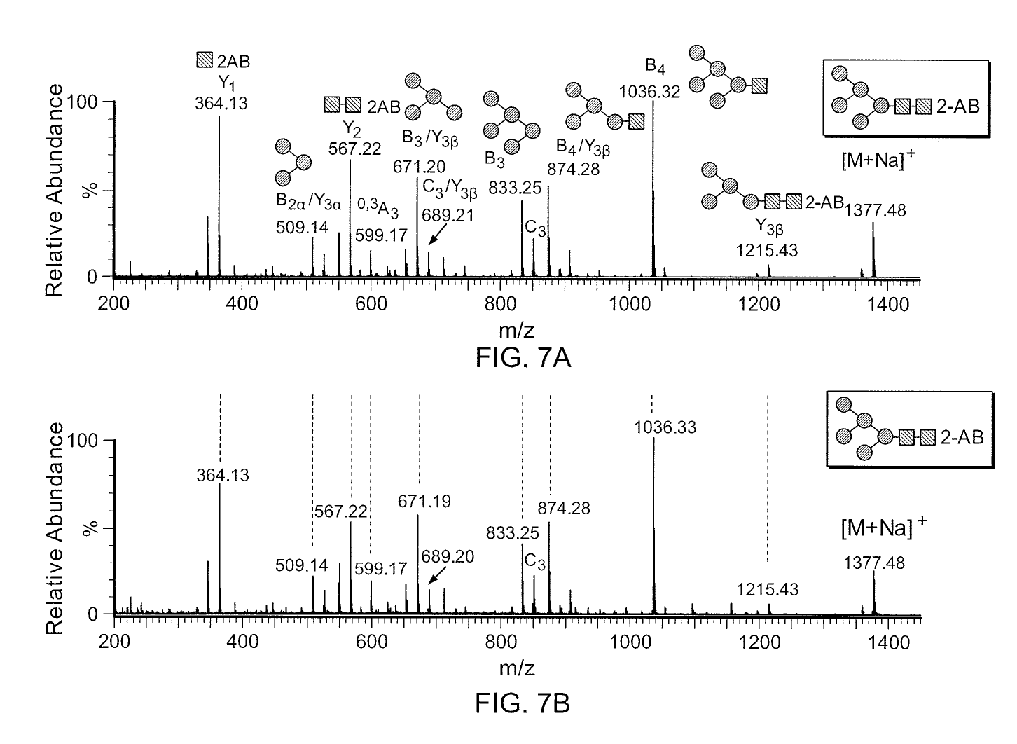

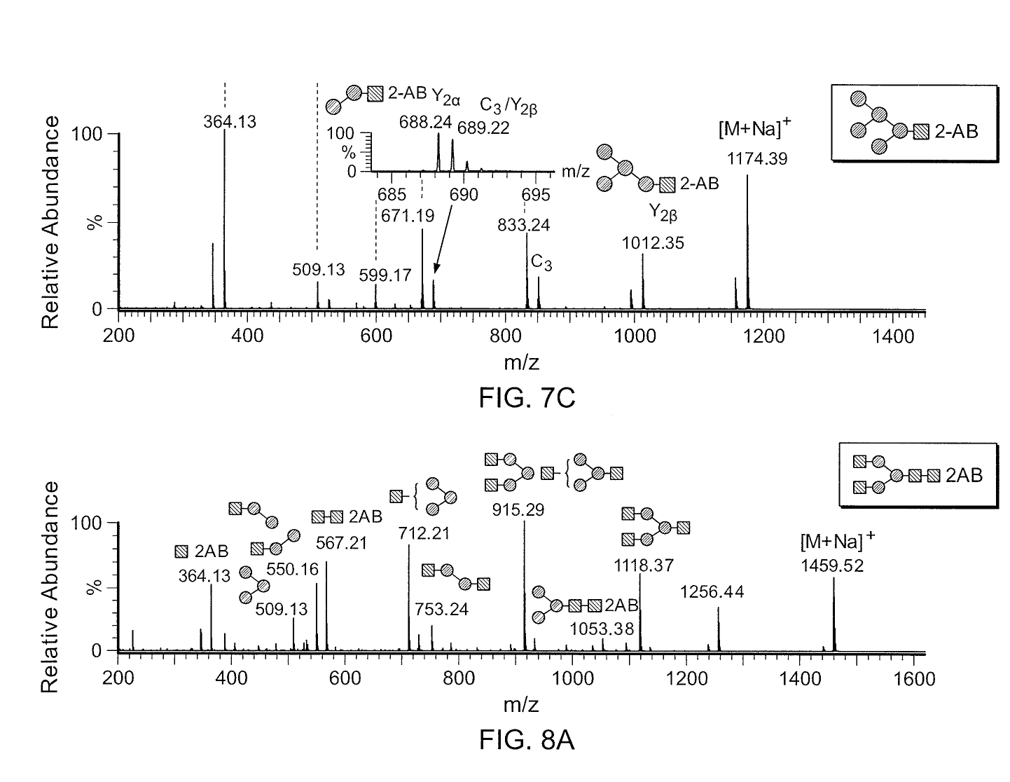

Structural characterization of the 1354.520 Da and 1151.446 Da species from the profiles of mAb N-glycans released with recombinant and native PNGase F was performed with MALDI-QTOF MS/MS. The MS/MS spectrum of a Man5 standard was compared with the MS/MS spectra of the collected 1354.520 Da and 1151.446 Da mAb N-glycan fractions (FIG. 7). Observation of the m/z 833.25 ion in all of the MS/MS spectra indicated that each carbohydrate comprised at least of five hexose residues (Scheme 1).

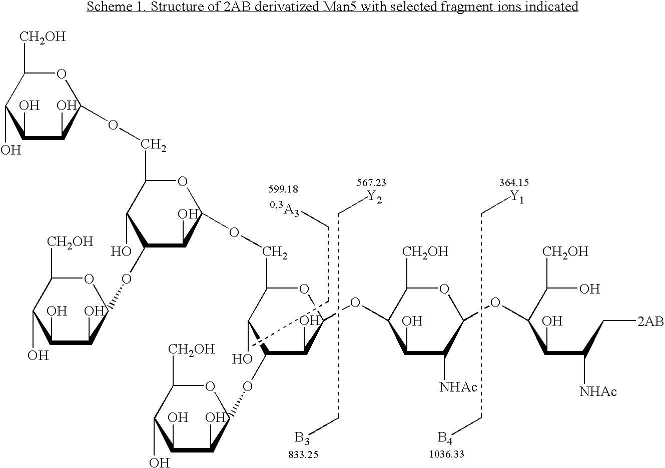

The presence of the ^{0,3}A₃ fragment ion (599.18 m/z) representing cross-ring cleavage of the core mannose residue verified that both mAb N-glycans had branching patterns consistent with that of the Man5 structure (Scheme 2). The similarity between the MS/MS spectra of the Man5 standard (FIG. 7A) and the nominally Man5 fraction (FIG. 7B) confirmed that the mAb N-glycan of mass 1354.520 Da was indeed Man5. All of the structural information in the MS/MS spectrum of the mAb N-glycan fraction nominally corresponding to Man5 with one rather than two GlcNAc residues supported the assignment of the 1151.446 Da glycan as Man₅GlcNAc (FIG. 7C). For example, the absence of a fragment ion at 567.21 m/z (Y₂ of Man5, Scheme 2) indicated that this carbohydrate had one rather than two GlcNAc residues on the reducing end. Likewise, the observation of an ion corresponding to 2AB derivatized Man4GlcNAc (1012.35 m/z) rather than underivatized Man₄GlcNAc (874.28 m/z) proved that this oligosaccharide has one terminal GlcNAc residue. The existence of an oligomannose structure missing a reducing terminal GlcNAc residue in samples treated with native PNGase F may be related to the presence of contaminant in the PNGase F preparation with enzymatic activity similar to that of Endo-β-N-acetylglucosaminidase H (Endo H) (R. B. Trimble, R. J. Trumbly, F. Maley, Endo-β-N-acetylglucosaminidase H from Streptomyces plicatus, Methods Enzymol. 138 (1987) 763-770). Since Endo H releases oligomannose structures from glycoproteins by cleaving the bond between the two GlcNAc reducing end residues of the carbohydrate, the Endo H released mAb N-glycans are missing a reducing end GlcNAc residue relative to the N-glycans present on the mAb and those released by PNGase F.

Other peaks were noted in the NP-HPLC profiles of mAb N-glycans with masses having one less GlcNAc residue than carbohydrates known to be present on the mAbs. Minor peaks with masses corresponding to G0 and G0F with three instead of four GlcNAc residues (G0-GlcNAc and G0F-GlcNAc) were observed in the NP-HPLC profiles of mAb N-glycans released with recombinant as well as native PNGase F (FIG. 6). These peaks are not likely to be artifacts of the PNGase F release since carbohydrate structures consistent with G0-GlcNAc (Man₃GlcNAc₃) and G0F-GlcNAc (Man₃GlcNAC₃Fuc) are part of the N-glycan biosynthetic pathway (R. Kornfield, S. Kornfield, Assembly of asparagine-linked oligosaccharides, Annu Rev. Biochem. 54 (1985) 631-664; J. B. Lowe, J. D. Marth, A genetic approach to mammalian glycan function, Annu Rev. Biochem. 72 (2003) 643-691) and masses for these carbohydrates were detected in the on-line RP-HPLC mass analysis of the intact glycosylated heavy chain subunit as well as tryptic glycopeptides. To verify that the nominally G0-GlcNAc and G0F-GlcNAc species have structures consistent with the natural complex-type N-glycans and are not missing a reducing terminal GlcNAc residue, fractions from the NP-HPLC profiles were collected and subjected to MALDI-QTOF MS/MS. Standards of Man₃GlcNAc₃ and Man₃GlcNAc₃Fuc were not available, thus, for purposes of knowledge-based characterization, the MS/MS spectra of the G0-GlcNAc and G0F-GlcNAc species were compared to those of the N-glycan standards, G0 (Man₃GlcNAc₄) and G0F (Man₃GlcNAc₄Fuc), respectively. By evaluating the MS/MS spectra of the “-GlcNAc” species relative to those of standards having core N-glycan structures such as G0 and G0F, it should be straightforward to determine whether the “-GlcNAc” species have core N-glycan structures or are missing a reducing terminal GlcNAc residue. The structural information obtained for G0-GlcNAc is discussed first, followed by the description of structural information obtained for G0F-GlcNAc.

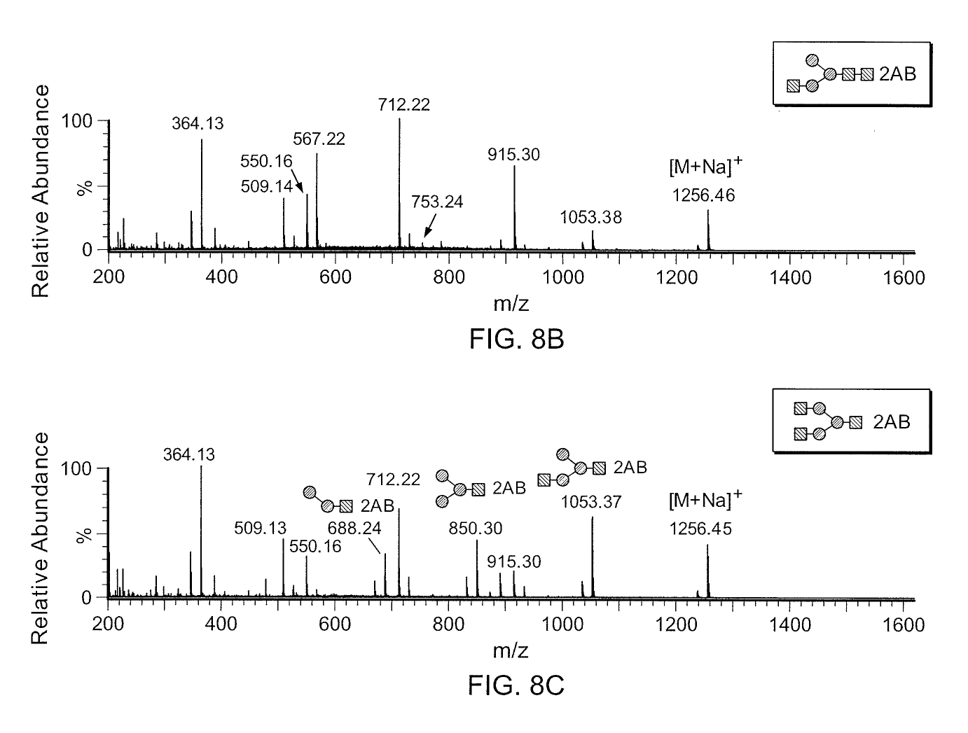

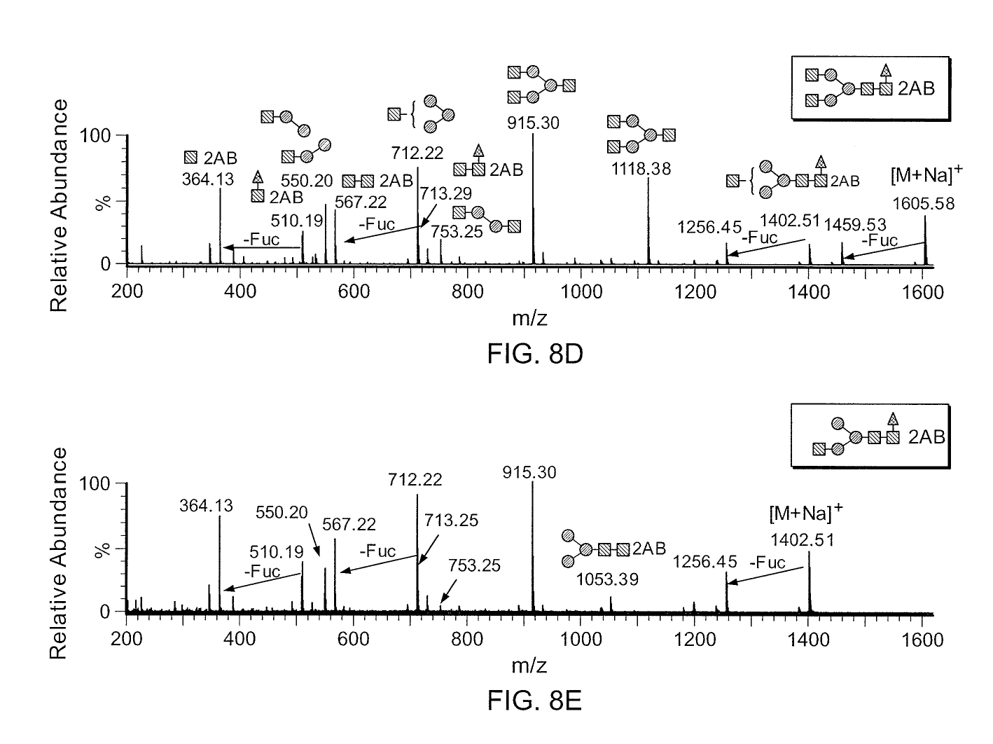

In the profiles of mAb N-glycans released with native PNGase F, peaks with masses corresponding to G0-GlcNAc were observed with retention times of 22.2 min. and 23.3 min. (FIGS. 6B and 6C). Both of these isomeric Man₃GlcNAc₃ peaks, referred to as G0-GlcNAc (22.2 min) and G0-GlcNAc (23.3 min.), were collected for structural analysis by MS/MS and the MS/MS spectra are shown in FIG. 8 along with the MS/MS spectrum of the G0 standard. When examining the differences in the MS/MS spectra of the Man₃GlcNAc₃ isomers (FIGS. 8B and 8C), the apparent differences in fragment ion masses in addition to differences in relative abundances of isobaric fragment ions indicated that the G0-GlcNAc isomers did not vary only in the position of a non-reducing terminal residue, as was the case for the G1F isomers. Instead, these isomers differed in the distribution of GlcNAc residues at the reducing and non-reducing termini. For example, the signature ion for a GlcNAc residue adjacent to the 2AB derivatized reducing terminal GlcNAc residue, m/z 567.22, was observed in FIG. 8A for the G0 standard and FIG. 8B for G0-GlcNAc (22.2 min), but not in FIG. 8C for G0-GlcNAc (23.3 min). This implied that the structure of the G0-GlcNAc (22.2 min) isomer had two of the three GlcNAc residues at the reducing end, while the G0-GlcNAc (23.3 min) isomer had one of the three GlcNAc residues at the reducing end. The presence of ions unique to the MS/MS spectrum of G0-GlcNAc (23.3 min) such as m/z 688.24, m/z 850.30, and m/z 1053.37 (FIG. 8C), also indicated that this carbohydrate had only one GlcNAc residue at the reducing end and that it was 2AB derivatized. In summary, MS/MS data provided evidence that the G0-GlcNAc (22.2 min) isomer has a single non-reducing GlcNAc residue and the G0-GlcNAc (23.3 min) isomer has non-reducing terminal GlcNAc residues on both the α(1,6) and α(1,3) arms. The low abundance of the m/z 753.24 ion in FIG. 8B appeared to indicate that the single GlcNAc was on the α(1,3) arm instead of the α(1,6) arm. Thus, the MS/MS structural information was consistent with the G0-GlcNAc (22.2 min) isomer representing a natural complex-type N-glycan structure and the G0-GlcNAc (23.3 min) isomer corresponding to a non-native oligosaccharide structure. This structure may have been generated by the removal of one of the two GlcNAc residues at the reducing end of the native complex-type G0 N-glycan prior to derivatization with 2AB. Given that the G0-GlcNAc (23.3 min) isomer has a non-native structure and is only observed in the NP-HPLC profiles of mAb glycans released with native PNGase F, it was considered an artifact of the mAb N-glycan release. This artifact may be related to the presence of a contaminating enzyme such as endo-β-N-Acetylglucosaminidase F2 (Endo F2) (J. H. Elder, S. Alexander, endo-β-N-Acetylglucosaminidase F: Endoglycosidase from Flavobacterium meningosepticum that cleaves both high-mannose and complex glycoproteins, Proc. Natl. Acad. Sci. USA 79 (1982) 4540-4544; R. B. Trimble, A. L. Tarentino, Identification of distinct endoglycosidase (Endo) activities in Flavobacterium meningosepticum: Endo F₁, Endo F₂, and Endo F₃, J. Biolog. Chem. 266 (1991) 1646-1651), which cleaves between the core reducing terminal GlcNAc residues of high mannose and biantennary carbohydrates and is isolated from the same species as the native PNGase F.

To complete the characterization of minor peaks observed in the NP-HPLC profiles of mAb N-glycans released with native and recombinant PNGase F (FIG. 6), the peak corresponding to G0F-GlcNAc was collected and subjected to MALDI-QTOF MS/MS. Since there was only one peak with a mass corresponding to that of G0F-GlcNAc observed in each of the fluorescence profiles shown in FIG. 6, it was believed to have a native structure rather than one missing a reducing terminal GlcNAc residue. This was confirmed by the data shown in the comparison of the MS/MS spectra of a commercially obtained G0F standard (FIG. 8D) with that of G0F-GlcNAc from a mAb N-glycan profile (FIG. 8E). The observation of the m/z 567.22 ion (FIG. 8E) verifies that the reducing end of the G0F-GlcNAc glycan has two GlcNAc residues, one of which is derivatized with 2AB. Based on the low abundance of the m/z 753.25 ion, the third GlcNAc residue of Man₃GlcNAc₃Fuc is on the α(1,3) arm rather than the α(1,6) arm. Thus, the MS/MS structural information in FIG. 8E indicated that the G0F-GlcNAc glycan had a core N-glycan structure with one non-reducing GlcNAc on the α(1,3) arm.

In summary of the investigation of variation observed in the low-level peaks in the NP-HPLC profiles of mAb N-glycans released with native and recombinant PNGase F, accurate mass and MS/MS structural information facilitated identification and subsequent verification of the identities of the glycans associated with these minor peaks. The source of the variation was traced to the use of native PNGase F, which may contain contaminating endoglycosidases. Because releasing the glycans with native PNGase F resulted in the observation of truncated structures such as Man5-GlcNAc and G0-GlcNAc with one instead of two reducing terminal GlcNAc residues, we changed our method to specify the use of recombinant PNGase F for mAb N-glycan release.

### Example 13

### Analysis of Glycan Structures Associated with rhBMP-2

### Release and Derivatization of N-Glycans

The recombinant human Bone Morphogenetic Protein-2 (rhBMP-2) samples were incubated with EndoH (New England Biolabs) overnight at 37° C. to release N-glycans. Released N-glycans were derivatized with 2AB in a manner similar to that published by Bigge et. al. The 2AB reagent was prepared by dissolving 47 mg 2AB and 63 mg of sodium cyanoborohydride in 1 mL of glacial acetic acid/dimethyl sulfoxide (30:70, v/v). A 10 μL aliquot of 2AB reagent was added to the EndoH reaction mixture after 16 h. The 2AB derivatization reaction proceeded for 2 h at 65° C. Then, the derivatization mixture was lyophilized using a Thermo Electron (Milford, Mass.) Speed Vac for approximately 1.5 h.

### Solid Phase Extraction (SPE) of 2AB Derivatized N-Glycans

Excess reagents from the N-glycan release and derivatization reactions were removed using 3 mL SupelClean NH₂ (Supelco, St. Louis, Mo.) SPE cartridges. The SPE loading, washing, and elution solutions were prepared using stock solutions of acetonitrile (A) and 250 mM ammonium formate, pH 4 (B). The loading solution was 80% A, 20% B (v/v), the wash solution was 65% A, 35% B (v/v), and the elution solution was 20% A, 80% B (v/v). The SPE vacuum manifold setting was 5 psi for all steps in the SPE process. Dried samples containing 2AB derivatized N-glycans were re-suspended in 100 μL loading solution. The SPE cartridge was conditioned with two 0.5 mL aliquots of the loading solution before the re-suspended derivatization mixture was loaded. After loading the sample, the cartridge was washed with two 1 mL aliquots of the wash solution and the derivatized glycans were eluted with three 0.6 mL aliquots of the elution solution. After lyophilzation of the eluent, the 2AB derivatized N-glycans were reconstituted with water (100 to 150 μL) prior to analysis by NP-HPLC.

### Normal Phase HPLC

Various 2AB derivatized high mannose N-glycan standards were used to optimize chromatographic separation and LC/MS performance. The 2AB derivatized N-glycans were profiled with a Waters (Milford, Mass.) 2695 Alliance HPLC system equipped with a Waters 2475 fluorescence detector (λ_{exc}=330 nm and λₑₘᵢₛₛ=420 nm). Additionally, a Waters 2487 dual-wavelength UV detector was used to monitor the absorbance at 330 nm and 214 nm. The NP-HPLC method employed a PolyLC (Columbia, Md.) polySulfoethyl Aspartamide column (4.6×100 mm, 5 μm) and a 1 mL/min gradient with mobile phases of acetonitrile (A) and water (B) (Table 1). The total run time was 65 min and the column temperature was maintained at 45° C. Typical injection volumes were 2 to 10 μL. For higher injection volumes, the sample solution composition was adjusted to 80% acetonitrile. For some mAb samples, chromatographically separated 2AB derivatized N-glycans were collected and lyophilized prior to further structural analysis by MALDI-QTOF MS/MS.

### On-Line NP-HPLC/MS

On-line NP-HPLC/MS experiments were performed with a Q-T of API US (Waters, Beverly, Mass.) mass spectrometer. Data was acquired and analyzed with MassLynx 3.5 software (Waters, Beverly, Mass.). The effluent from the HPLC system was split postcolumn so that ˜100 μL/min was directed to the ESI source of the mass spectrometer. All experiments were performed in the positive ion mode with an ESI voltage of 3.0 kV, ion source cone voltage of 25 V, and collision energy (CE) of 5 eV. Data was acquired from m/z 50 to m/z 3000 in 2 s scans with 0.1 s interscan delay. Sodium iodide was used to calibrate the instrument, and during calibration the ion source temperature was 80° C. and the desolvation temperature was 120° C. After calibration, the dynamic calibration temperature compensation circuitry was activated for LC/MS, which is performed with higher temperatures (115° C. and 275° C., respectively).

The contents of all references cited herein are incorporated by reference in their entirety.

A number of embodiments of the invention have been described. Nevertheless, it will be understood that various modifications may be made without departing from the spirit and scope of the invention. Accordingly, other embodiments are within the scope of the following claims.

What is claimed:

1. A method of evaluating or processing a glycan structure or structures of a glycomolecule preparation, comprising:
subjecting a glycan or glycans from a glycomolecule preparation to high performance liquid chromatography (HPLC) in the absence of an ion pairing agent to evaluate a glycan structure or structures, and
subjecting the HPLC-evaluated glycan or glycans to mass spectrometry to further evaluate a glycan structure or structures,
to thereby evaluate or process the glycan structure or structures of a glycomolecule preparation.

2. The method of claim 1, wherein the glycan structure or structures is an N-linked glycan.

3. the method of claim 1, wherein the glycan structure or structures is an O-linked glycan.

4. The method of claim 2, wherein the glycan structure or structures is one or more of: a sialylated, complex glycan, a non-sialylated complex glycan, a high mannose glycan and a hybrid glycan.

5. The method of claim 4, wherein one or more of the glycan structures is a sialylated, complex glycan and the glycan is a bisialylated glycan, a monosialylated glycan, or combinations thereof.

6. The method of claim 4, wherein the sialylated complex glycan or non-sialylated complex glycan is biantennnary.

7. The method of claim 1, wherein the glycomolecule preparation is selected from the group consisting of a glycoprotein preparation and a glycolipid preparation.

8. The method of claim 7, wherein the glycomolecule is a glycoprotein and the glycoprotein is an antibody or antigen binding fragment thereof.

9. The method of claim 1, wherein the HPLC-evaluated glycan or glycans are not subjected to one or more of ion pairing removal, desalting, dialysis and drying prior to evaluation with mass spectrometry.

10. The method of claim 1, wherein the HPLC is selected from normal phase HPLC (NP-HPLC) and reverse phase HPLC (RP-HPLC).

11. The method of claim 10, wherein the HPLC is NP-HPLC.

12. The method of claim 1, wherein the glycan or glycans are subjected to HPLC with a mobile phase of acetonitrile, water or a combination thereof.

13. the method of claim 12, wherein the glycan or glycans are subjected to HPLC with a mobile phase of acetonitrile and water.

14. The method of claim 11, wherein the HPLC is hydrophilic interaction chromatography.

15. The method of claim 14, wherein the HPLC uses a polySulfoethyl Aspartamide (polySEA) column or a SeQuant column.

16. The method of claim 1, wherein the mass spectrometry is one or more of: electrospray ionization mass spectrometry (ESI-MS), turbospray ionization mass spectrometry, nanospray ionization mass spectrometry, thermospray ionization mass spectrometry, sonic spray ionization mass spectrometry, surface enhanced laser desorption ionization mass spectrometry (SELDI-MS) and matrix assisted laser desorption/ionization mass spectrometry (MALDI-MS).

17. The methods of claim 16, wherein the mass spectrometry is ESI-MS or MALDI-MS.

18. The method of claim 1, wherein the evaluation by mass spectrometry uses a quadrupole mass analyzer, a time of flight (TOF) mass analyzer or a hybrid quadrupole/linear ion trap mass analyzer.

19. The method of claim 1, wherein glycan structure or structures are analyzed for branching, linkages between monosaccharides and location of monosaccharides.

20. The method of claim 1, wherein the glycomolecule preparation is evaluated for the presence or quantity of: a fucosylated biantennary complex glycan having no reducing end terminal galactose residues, a fucosylated biantennary complex glycan having one reducing end terminal galactose residue, a fucosylated biantennary complex glycan having two reducing end terminal galactose residues, a biantennary complex glycan having no reducing end terminal galactose residues, a biantennary complex glycan having one reducing end terminal galactose residue, a biantennary complex glycan having two reducing end terminal galactose residues, a fucosylated biantennary complex glycan having two galactose residues and one N-acetylneuraminic acid residue, a fucosylated biantennary complex glycan having two galactose residues and two N-acetylneuraminic acid residues, a biantennary complex glycan having two galactose residues and two N-acetylneuraminic acid residues, a high mannose glycan having five mannose residues, a high mannose glycan having six mannose residues, a high mannose glycan having seven mannose residues, a high mannose glycan having eight mannose residues, and a high mannose glycan having nine mannose residues.

21. The method of claim 1, wherein the method includes evaluating a value for a glycan structure or structures from the HPLC-evaluated glycan or glycans to determine if the value meets a reference standard.

22. The method of claim 21, wherein the value is the presence or amount of a glycan structure or structures.

23. The method of claim 1, wherein the method includes evaluating a value for a glycan structure or structure from the MS-evaluated glycan or glycans to determine if the value meets a reference standard.

24. The method of claim 23, wherein the value is the presence of a glycan structure or structures.

25. The method of claim 21, wherein the method further includes evaluating a value for a glycan structure or structure from the MS-evaluated glycan or glycans to determine if the value meets a reference standard.

26. The method of claim 25, wherein the value is the presence of a glycan structure or structures.

27. The method of claim 21, 23 or 25, wherein the reference standard is a release specification, a label requirement, or a compendia specification.

28. The method of claim 21, 23 or 25, wherein the reference standard is a different preparation of the glycomolecule.

29. The method of claim 28, wherein the reference standard is a different preparation of the glycomolecule made by a different method than the glycomolecule being evaluated.

30. The method of claim 1, wherein the method further includes making a decision about the glycomolecule preparation based upon the analysis.

31. The method of claim 30, wherein the decision includes one or more of: accepting or discarding the preparation, releasing or withholding the preparation, formulating the preparation, packaging the preparation, labeling the preparation, shipping, relocating, selling or offering to sell the preparation.

32. The method of claim 1, wherein prior to HPLC the glycan or glycans are removed from the glycomolecule.

33. The method of claim 32, wherein the glycan or glycans are removed enzymatically.

34. The method of claim 33, wherein the glycan or glycans are N-linked and the enzyme is PNGase F.

35. The method of claim 1, wherein the glycomolecule is a glycoprotein and the glycoprotein preparation has further been evaluated by peptide mapping or peptide sequencing.

36. The method of claim 35, wherein the glycoprotein has been digested and the digested peptide fragments have been evaluated using HPLC, e.g., RP-HPLC, and mass spectrometry, ESI-MS.

37. The method of claim 35, wherein the glycoprotein has been digested and the digested peptide fragments have been evaluated using MS/MS, e.g., nanoESI-q-TOF MS/MS.

38. The method of claim 36, wherein the glycoprotein has been digested by reduction/alkylation and proteolysis.

39. The method of claim 1, wherein the glycomolecule is a glycoprotein and the glycoprotein preparation has further been evaluated by intact and subunit analysis.

40. The method of claim 35, wherein the glycan has been removed from the glycoprotein and the protein has been evaluated using HPLC, e.g., RP-HPLC, and mass spectrometry, e.g., ESI-q-TOF MS.

41. The method of claim 35, wherein the glycan has been removed from the glycoprotein and the protein has been evaluated using ESI-MS, e.g., nanoESI-q-TOF.

42. The method of claim 41, wherein the glycan is an N-glycan and the N-glycan is removed using PNGase F.

43. The method of claim 40, wherein the glycoprotein is an antibody and heavy chain and light chain subunits of the glycoprotein preparation are separated, e.g., using reduction and alkylation.

44. The method of claim 43, wherein the heavy chain and/or light chain subunits of the glycoprotein preparation have been evaluated using HPLC, e.g., RP-HPLC, and mass spectrometry, e.g., ESI-q-TOF MS.

45. The method of claim 43, wherein the heavy chain and/or light chain subunits of the glycoprotein preparation have been evaluated using ESI-MS, e.g., nanoESI-q-TOF.

46. The method of claim 1, wherein the glycomolecule preparation is a test batch and the test batch can be evaluated to determine if the test preparation is expected to have one or more properties of a commercially available version of the glycomolecule preparation.

47. A method of evaluating or processing a glycomolecule preparation, comprising:
making a determination about a glycomolecule preparation based upon the method of claim 1.

48. The method of claim 47, wherein the method further comprises: accepting or discarding the preparation, releasing or withholding the preparation, formulating the preparation, packaging the preparation, labeling the preparation, shipping, relocating, selling or offering to sell the preparation, based upon the determination.

49. A method of evaluating a glycomolecule preparation for a biological activity, comprising
proving an evaluation of a glycan structure or structures of the glycomolecule preparation obtained by the method of claim 1, and
comparing the evaluation of the glycan structure or structures to an evaluation obtained by the method of claim 1 on a second glycomolecule preparation,
making a determination regarding biological activity of the glycoprotein preparation based upon similarities or differences in the glycan structure or structures of the glycomolecule preparation and the second glycomolecule preparation.

50. The method of claim 49, wherein the glycan structure or structures are a direct measure of the biological activity.

51. The method of claim 49, wherein the glycan structure or structures are an indirect measure of biological activity.

52. The method of claim 49, wherein the biological activity is selected from immunogenecity, half life, stability, clearance and binding.

53. A method of evaluating the effect of glycan structures or structures on a biological activity of a glycomolecule, comprising:
providing a first glycomolecule preparation having a first activity or level of an activity and a second glycomolecule preparation that does not have the activity or has a different level of the activity,
providing an evaluation of glycan structure of the first glycomolecule preparation and the second glycomolecule preparation obtained by the method of claim 1, determining the absence or presence of differences in the glycan structure or structures of the first and second preparation, to thereby evaluate the effect of the glycan structure or structures on the activity.

54. A method of analyzing a process, e.g., a manufacturing process, of a glycomolecule preparation, comprising:
providing a glycomolecule preparation made by a selected process,
analyzing a value for a glycan structure or structures of the glycomolecule preparation by a method described herein,
comparing the value to a reference standard, to thereby evaluate the process.

55. The method of claim 54, wherein the glycomolecule preparation is prepared by the same process as the process used to obtain the glycomolecule preparation or preparations used to obtain the reference standard.

56. The method of claim 54, wherein the glycomolecule preparation is made by a different process than the glycomolecule preparation or preparations used to obtain the reference standard.

57. The method of claim 54, wherein the method further comprises maintaining the manufacturing process based, at least in part, upon the analysis.

58. The method of claim 54, wherein the method further comprises altering the manufacturing process based, at least in part, upon the analysis.
